(19) United States
(12) Patent Application Publication
(10) Pub. No.: US 2024/0173431 A1
(43) Pub. Date: May 30, 2024

(54) NANOTHERAPEUTICS FOR TREATMENT OF SARS-COV-2

(71) Applicant: OREGON STATE UNIVERSITY, Corvallis, OR (US)

(72) Inventors: Jeonghwan KIM, Corvallis, OR (US); Anindit MUKHERJEE, Corvallis, OR (US); Gaurav SAHAY, Corvallis, OR (US)

(21) Appl. No.: 18/281,122

(22) PCT Filed: Mar. 10, 2022
(86) PCT No.: PCT/US2022/019705

§ 371 Date: Sep. 8, 2023

Related U.S. Application Data
(60) Provisional application No. 63/159,032, filed on Mar. 10, 2021.

(51) Int. Cl. A61K 48/00; A61K 9/00; A61K 9/127; A61K 9/51; A61K 38/48; A61P 31/14

(52) U.S. Cl. CPC A61K 48/005; A61K 9/007; A61K 9/1271; A61K 9/5123; A61K 38/4813; A61P 31/14; C12Y 304/17023

(57) ABSTRACT

Embodiments provide for therapeutic agents and methods of use thereof, for treating one or more diseases or conditions. In one example, the therapeutic agent is a lipid nanoparticle comprised of an ionizable lipid, a PEG lipid, a sterol, and a structural lipid, the lipid nanoparticle including one or more mRNA molecules encoding for a soluble form of human angiotensin-converting enzyme 2 and/or variations thereof. In this way, one or more signs or symptoms of Covid-19 may be treated via use of the therapeutic agent.

### CROSS REFERENCE TO RELATED APPLICATION

The present application claims priority to U.S. Provisional Patent Application No. 63/159,032, which was filed Mar. 10, 2021, the disclosure of which is hereby incorporated by reference.

### ACKNOWLEDGEMENT OF GOVERNMENT SUPPORT

This invention was made with government support under 1R01HL146736-01, awarded by the National Institutes of Health. The government has certain rights to the invention.

### REFERENCE TO A SEQUENCE LISTING

This application incorporates by reference the Sequence Listing submitted in Computer Readable Form as file P017Z_261212, created on Mar. 4, 2021 and containing 34,093 bytes.

### TECHNICAL FIELD

Embodiments herein relate to treating illness with gene therapy, and more specifically, to nanoparticle delivery of mRNA encoding one or more gene products into subjects afflicted with a coronavirus.

### BACKGROUND

Severe acute respiratory syndrome coronavirus 2 (SARS-COV-2) enters through the airways and infects the lungs, causing lethal pulmonary damage in vulnerable patients. This virus contains spike proteins on its envelope that binds to human angiotensin-converting enzyme 2 (hACE2) expressed on the surface of airway cells, enabling entry of the virus for causing infection. In severe cases, the virus enters the circulatory system, contributing to multi-organ failure. Remdesivir, an investigational antiviral drug, has shown encouraging evidence in improving time of recovery among patients. The overall mortality rate, however, remains unchanged while conflicting reports have emerged on clinical outcomes. Dexamethasone, an anti-inflammatory steroid repurposed for COVID-19, was shown to lower mortality in the patients when used in conjunction with respiratory support. Further treatments for SARS-COV-2 that can be rapidly prepared and readily distributed represent an urgent need to reduce the adverse impact of SARS-COV-2 on human health and the global economy.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1A illustratively depicts a rationale for soluble ACE2 (hsACE2) messenger RNA (mRNA) therapeutics in treating SARS-COV-2 infection;

FIG. 1B is a schematic showing potential routes of administration for a soluble ACE2 mRNA therapeutic of the present disclosure;

FIG. 1C is a schematic of in-vitro-transcribed mRNA (IVT mRNA) encoding hsACE2 variant protein, TEV site, and v5 tag. Also depicted is a comparison of endogenous ACE2 and the hsACE2 variant of the present disclosure;

FIG. 1D is a schematic illustration of a lipid nanoparticle (LNP) of the present disclosure encapsulating mRNA;

FIGS. 2A-2B are western blot images of cell-free media (FIG. 2A) and cell lysates (FIG. 2B) derived from 293T cell cultures transfected with hsACE2 mRNA using lipofectamine 3000 for 24 hours;

FIGS. 2C-2D are graphs showing representative data of size distribution and RNA encapsulation of LNP/hsACE2 (FIG. 2C) and eLNP/hsACE2 (FIG. 2D) used in the present disclosure;

FIGS. 3A-3B are graphs showing in vitro luciferase (FIG. 3A) and cell viability (FIG. 3B) assays of 293T cells transfected with LNP/Fluc or eLNP/Fluc for 24 hours (10-200 ng mRNA per well, n=6). At FIG. 3A, the ordering is LNP (left) and eLNP (right) for each dosage shown. Statistical analysis was performed using Student's t test. ** p<0.01, *** p<0.001, **** p<0.0001. All data were expressed as the mean±S.D .;

FIGS. 3C-3D depict western blot images with cell-free media (FIG. 3C) and cell lysates (FIG. 3D) of 293T cells after mRNA transfection using various LNPs. Treatment and mRNA dose are described on the right of each blot;

FIG. 3E is a graph showing expression of hsACE2 protein in the 293T cell lysates of FIG. 3D normalized to the expression of B-actin by densitometry;

FIGS. 3F-3G illustrate that production of hsACE2 protein is dependent on mRNA dosage. FIG. 3F is a western blot of cell-free conditioned media from 293T cells after mRNA transfection using various LNPs. Treatment and mRNA dose are described under each blot (Duplicate (D): n=2, Triplicate (T): n=3). The blot of FIG. 3F was stained with Coomassie blue to visualize total protein in FIG. 3G;

FIGS. 4A-4B are graphs illustrating in vitro luciferase (FIG. 4A) and cell viability (FIG. 4B) assays of Hep G2 cells transfected with LNP/Fluc or eLNP/Fluc for 24 h (10-200 ng mRNA per well, n=6). At FIG. 4A, the ordering is LNP (left) and eLNP (right) for each dosage shown. Statistical analysis was performed using Student's t test. ** p<0.01, *** p<0.001, **** p<0.0001. All data were expressed as the mean±S.D .;

FIGS. 4C-4D illustrate that production of hsACE2 protein is dependent on mRNA dosage. FIG. 4C is a western blot of cell-free conditioned media from Hep G2 cells after mRNA transfection using various LNPs. Treatment and mRNA dose are described under each blot (Duplicate (D): n=2, Triplicate (T): n=3). The blot of FIG. 4C was stained with Coomassie blue to visualize total protein in FIG. 4D;

FIGS. 4E-4F illustrate western blot data (FIG. 4E) and quantitation (FIG. 4F) of expression of hsACE2 protein in Hep G2 cell lysates compared to expression of β-actin. FIG. 4E shows a western blot of cell lysates of Hep G2 cells after mRNA transfection using various LNPs. Treatment and mRNA dose are described on the right of each blot (T; n=3). FIG. 4F is a graph showing expression of hsACE2 protein in the Hep G2 cell lysates normalized to the expression of B-actin by densitometry. Statistical analysis was performed using Student's t test. ** p<0.01, *** p<0.001, **** p<0.000122 All data were expressed as the mean±S.D .;

FIG. 5A are in vivo bioluminescent images of BALB/c mice after treatment of 0.05 mg/kg mRNA delivered through IV injection of LNP/Fluc or eLNP/Fluc (n=5);

FIG. 5B is a graph showing quantification of bioluminescent signals from the images of FIG. 5A. Region of interest was kept constant in all images (n=5). The order at FIG. 5B is LNP (left), eLNP (right) for each time (h) shown. Statistical analysis was performed using Student's t test. ** p<0.01, *** p<0.001, **** p<0.0001;

FIGS. 5C-5D depict an image of western blot data (FIG. 5C) with mouse sera collected with predetermined time intervals after IV injection of 0.15 mg/kg eLNP/hsACE2, and densitometric quantitation (FIG. 5D) of temporal levels of the circulatory hsACE2 protein in mouse sera after IV injection of eLNP/hsACE2 (n=5). Expression of hsACE2 protein was normalized to the total amount of protein in each lane. All data were expressed as the mean±S.D.;

FIGS. 5E-5F depict an image of western blot data (FIG. 5E) with mouse sera collected at predetermined time intervals after IV injection of eLNP/hsACE2 (n=5), and quantification of the temporal level (FIG. 5F) of the circulatory hsACE2 protein in each mouse (e.g., mouse 1 or M1, mouse 2 or M2, mouse 3 or M3, mouse 4 or M4, and mouse 5 or M5). Expression of the hsACE2 protein was normalized to the total amount of protein in each lane;

FIG. 5G depicts an image of each of the blots of FIG. 5B stained with Coomasie blue for visualization of total protein in each lane as described;

FIG. 5H depicts a western blot of hsACE2 protein in mouse sera after IV injection of eLNP/hsACE2;

FIG. 5I-5J depict a western blot (FIG. 5I) of liver homogenates collected from BALB/c mice after IV injection of 0.15 mg/kg eLNP/hsACE2, and expression (FIG. 5J) of hsACE2 protein in mouse liver homogenates after IV injection of eLNP/hsACE2 (n=4). Densitometric analysis of hsACE2 protein expression normalized to β-actin levels. Statistical analysis was performed using Student's t test. ** p<0.01, *** p<0.001, **** p<0.0001. All data were expressed as the mean±S.D.;

FIGS. 6A-6B are graphs showing in vitro transfection of Calu-3 by 48-hour incubation of LNP/Fluc or eLNP/Fluc at various mRNA doses (n=6) (FIG. 6A), and cell viability of Calu-3 cells transfected with LNP/Fluc or eLNP/Fluc for 48 h (10-200 ng mRNA per well, n=6) (FIG. 6B). Statistical analysis was performed using Student's t test. **** p<0.0001. All data were expressed as the mean±S.D. At FIG. 6A, the ordering is LNP (left) and eLNP (right) for each dosage shown;

FIG. 6C is a western blot of cell-free media of Calu-3 cell culture after hsACE2 mRNA transfection using LNPs. Treatment and mRNA dose are described under each blot (Q: n=4);

FIGS. 6D-6E depict a western blot (FIG. 6D) of cell lysates of Calu-3 cells after mRNA transfection using LNP/hsACE2 and eLNP/hsACE2 (n=4), and a graph (FIG. 6E) illustrating expression of hsACE2 protein in the Calu-3 cell lysates normalized to the expression of B-actin by densitometry. Statistical analysis was performed using Student's t test. ** p<0.01. All data were expressed as the mean±S.D.;

FIG. 6F is an image of the blot of FIG. 6C stained with Coomassie blue to visualize total protein;

FIGS. 6G-6H depict bioluminescent images of BALB/c mice at 48 hours after treatment of 0.5 mg/kg mRNA delivered through intratracheal instillation of eLNP/Fluc. FIG. 6G shows in vivo and FIG. 6H shows ex vivo images of luciferase expression;

FIG. 61 depicts a western blot of hsACE2 protein in the bronchoalveolar lavage fluid (BALF). BALB/c mice were untreated or transfected with hsACE2 mRNA at 0.75 mg/kg mRNA through intratracheal instillation of eLNP/hsACE2, and the BALF was harvested after 24 h or 48 h post-administration (n=3). For enrichment of the hsACE2 protein, BALF was subjected to the immunoprecipitation using anti-V5 antibody prior to western blot;

FIG. 7A is a table showing that cell-free media from untreated or hsACE2 transfected 293T cell culture were incubated in the presence or absence of the RBD of the SARS-COV-2 prior to co-immunoprecipitation (co-IP). +and − define the presence and absence of the treatment, respectively;

FIG. 7B illustratively depicts a schematic workflow of co-immunoprecipitation as pertains to the present disclosure;

FIGS. 7C-7D show results of co-immunoprecipitation experiments using anti-V5 tag antibody. Upper and lower blots were probed using anti-V5 tag (for hsACE2) and anti-His tag (for RBD) antibodies, respectively. After co-IP, eluted samples (FIG. 7C) and flow-through samples (FIG. 7D) were analyzed using western blot;

FIGS. 7E-7F show results of co-immunoprecipitation experiments using anti-His tag antibody. Upper and lower blots were probed using anti-V5 tag (for hsACE2) and anti-His tag (for RBD) antibodies, respectively. After co-IP, eluted samples (FIG. 7E) and flow-through samples (FIG. 7F) were analyzed using western blot;

FIG. 7G is an image of a western blot of hACE2 in various cell lysates as shown;

FIG. 7H are graphs showing titration of various pseudovirus in (left) 293T cells and (right) 293T-hACE2 cells (n=3). Spike pseudovirus 705, pseudovirus with no envelope 710, VSV-G pseudovirus 715. Normalized luciferase expressions of VSV-G pseudovirus at 1 and 3-fold-dilutions were not presented due to saturation of signal. All data were expressed as the mean±S.D .; and

FIGS. 7I-7J are graphs showing normalized luciferase expression corresponding to Fluc-packaged lentivirus pseudotyped with (FIG. 7I) the spike protein of SARS-COV-2 or (FIG. 7J) VSV-G incubated with or without hsACE2 protein in 293T-hACE2 cells. Pseudoviruses were serially diluted for treatment and normalized luciferase expression was measured. All data were expressed as the mean±S.D. For both FIGS. 7I-7J, the order of the graphs are VSV-G alone (left) and hsACE2+VSV-G (right) for each fold-dilution.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Terms

Administration: To provide or give a subject one or more agents, such as an mRNA agent alone or included within a delivery vehicle such as a lipid nanoparticle (LNP) that treats one or more symptoms associated with a condition/disorder or disease including but not limited to viral infection/immune response to antigen, hypertension, stroke, or any disease or condition at least partly due to dysregulation of the Renin Angiotensin Aldosterone System (RAAS) by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Agent: Any protein, nucleic acid molecule (including chemically modified nucleic acids), compound, antibody, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response.

Contacting: Placement in direct physical association, including both a solid and liquid form. Contacting an agent with a cell can occur in vitro by adding the agent to isolated cells or in vivo by administering the agent to a subject.

Effective amount: An amount of agent that is sufficient to generate a desired response, such as reducing or inhibiting one or more signs or symptoms associated with a condition or disease (e.g., COVID-19 caused by infection with SARS-CoV-2). When administered to a subject, a dosage will generally be used that will achieve target tissue/cell/bloodstream concentrations. In some examples, an “effective amount” is one that treats one or more symptoms and/or underlying causes of any of a disorder or disease. In a representative example, an “effective amount” is a therapeutically effective amount in which the agent alone or with an additional therapeutic agent(s), induces the desired response such as reduction in one or more symptoms associated with COVID-19 or other coronavirus.

The symptoms and/or underlying cause of a disease, syndrome, viral infection, etc., do not need to be completely inhibited for the pharmaceutical preparation to be effective. For example, a pharmaceutical preparation may decrease the progression of the disease, syndrome, viral infection, etc., by a desired amount, for example by at least 10%, at least 20%, at least 50%, at least 60%, at least 70%, at least 80%, at least 90%, at least 95%, at least 98%, or even at least 100%, as compared to the progression typical in the absence of the pharmaceutical preparation.

The disclosed therapeutic agents can be administered in a single dose, or in several doses, for example hourly, daily, weekly, monthly, yearly, during a course of treatment. The effective amount can be dependent on the subject being treated, the severity and type of the condition being treated, and the manner of administration.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA (mRNA) to protein. Regulation can include controls on transcription, translation, RNA transport and processing, degradation of intermediary molecules such as mRNA, or through activation, inactivation, compartmentalization or degradation of specific protein molecules after they are produced. In an example, expression, such as expression of a soluble form of angiotensin-converting enzyme 2 (ACE2), can be regulated to treat one or more signs or symptoms associated with viral infection, hypertension, etc., as discussed herein.

The expression of a nucleic acid molecule can be altered relative to a normal (wild type) nucleic acid molecule. Alterations in gene expression, such as differential expression, include but are not limited to: (1) overexpression; (2) underexpression; or (3) suppression of expression. Alterations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it nominally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Luciferase: A generic term for a class of oxidative enzymes that produce bioluminescence. Found naturally in insect fireflies and in luminous marine and terrestrial microorganisms, luciferase is thus a light-producing enzyme. When expressed in mammalian or insect cells, the native signal sequences of these luciferases are functionally active, mediating their export from within the cell to the surrounding culture medium. Bioluminescence assays are conducted using culture media, whereupon the activity of the secreted luciferases provides a readout of the biological signaling event under study.

Patient: As used herein, the term “patient” includes human and non-human animals. The preferred patient for treatment is a human. “Patient” and “subject” are used interchangeably herein.

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more 001 modulatory agents.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

Preventing, treating or ameliorating a disease: “Preventing” a condition/disease (such as COVID-19) refers to inhibiting the full development of a disease. “Treating” refers to a therapeutic intervention that ameliorates a sign or symptom of a condition/disease or pathological condition after it has begun to develop. “Ameliorating” refers to the reduction in the number or severity of signs or symptoms of a condition/disease.

Treating a disease: A therapeutic intervention that ameliorates a sign or symptom of a condition/disease or pathological condition including but not limited to an infection by a coronavirus, such as a sign or symptom of COVID-19. Treatment can induce remission or cure of a condition or slow progression, for example, in some instances can include inhibiting the full development of a disease, for example preventing development of adverse conditions associated with COVID-19. Prevention of a disease does not require a total absence of disease. For example, a decrease of at least 50%, or at least 40%, or at least 30%, or at least 20% can be sufficient.

Treating a condition/disease can be a reduction in severity of some or all clinical symptoms of the disease or condition, a reduction in the number of relapses of the disease or condition, an improvement in the overall health or well-being of the subject, by other parameters well known in the art that are specific to the particular disease or condition, and combinations of such factors. It may be understood that treating a disease as discussed is not limited to viral infection and hypertension, but can include others (e.g., other conditions/diseases where dysregulation of RAAS is involved, cancer, and the like) as disclosed herein.

Under conditions sufficient for: A phrase that is used to describe any environment that permits the desired activity. One example includes administering a disclosed agent to a subject under conditions sufficient to allow the desired activity. In particular examples, the desired activity is increasing the expression or activity of a soluble form of human ACE2 (hsACE2).

Wild-type: A strain, gene or characteristic which prevails among individuals in natural conditions, as distinct from an atypical mutant type.

### II. Pharmaceutical Agents and Methods of use Thereof

SARS-COV-2, the pathogen of coronavirus disease 2019 (COVID-19), is a B-coronavirus that primarily enters through the airways and lungs. The envelope of SARS-COV-2 is decorated with homotrimeric spike (S) proteins that bind to the human angiotensin-converting enzyme 2 (hACE2) receptor expressed on the cell surface. The S protein is composed of S1 and S2 subunits responsible for viral attachment and fusion, respectively. Binding between the receptor-binding domain (RBD), which is located within the S1 subunit, and hACE2 triggers a cascade that accelerates cellular entry and viral membrane fusion. hACE2 is expressed in the lungs, heart, kidney, and intestine.

hACE2 functions as a key enzyme that participates in the Renin Angiotensin Aldosterone System (RAAS) responsible to maintain blood pressure. hACE2 is a carboxypeptidase that converts Angiotensin 1 to Angiotensin (1-9) or Angiotensin II to Angiotensin (1-7), both of which are vasodilators with cardioprotective effects through regulation of blood pressure. SARS-COV-2 interacts with hACE2 to enter and infect human airway epithelial cells, causing cytotoxic responses. It also can lead to development of pneumonia and cytokine storm, resulting in Acute Respiratory Distress Syndrome (ARDS) in severe cases. Once the virus infiltrates systemic circulation, it can dysregulate RAAS and immune system, cause endothelial cell damage, possibly target other tissues that express hACE2, and overall cause a multiorgan failure.

hACE2 consists of three segments: an extracellular segment that contains the peptidase domain where the RBD binds to, a transmembrane segment, and an intracellular segment. hACE2 can be cleaved by peptidases at the neck region of the extracellular segment, releasing a soluble form of hACE2 (hsACE2) which is enzymatically active. Since the RBD of SARS-COV-2 binds to the extracellular domain of hACE2, hsACE2 protein may be capable of reducing the viral infection through competitive inhibition. However, it is herein recognized that a relatively short half-life of the recombinant hsACE2 in the bloodstream would undesirably necessitate repeated administrations to ensure long-term circulation of the protein for days after exposure to SARS-COV-2. A short half-life of soluble ACE2 (<2 h in mouse) thus severely limits its time window of action and extended residence of hsACE2 is desirable to mitigate SARS-COV-2 mediated RAAS activation and hence to reduce inflammation-related injury of organs.

To overcome these challenges, herein disclosed is the use of LNPs to deliver in-vitro-transcribed messenger RNA (IVT mRNA) for rapid expression of hsACE2. This strategy may allow for rapid clearance of the captured virus while maintaining hsACE2 levels that can surveil circulation, clear the virus, and rescue the disrupted RAAS system. LNP-delivered mRNA as herein disclosed provides a transient yet high expression of protein with proper folding and post-translational modifications, but without risk of insertional mutagenesis as is associated with viral-based gene therapy. Unlike viral vectors, this platform technology can be repeatedly administered to sustain protein production until the infection subsides and cease of the treatment allows for clearance of hsACE2 within days, mitigating any off-target effects. In this way, expression of hsACE2 may prevent SARS-COV-2 from binding to cell surface receptors and block its entry. Discussed herein, an IVT mRNA was designed to encode the 1-740 amino acid sequence of hACE2 with a cleavable V5-epitope tag at the C-terminus.

Specifically, it is herein disclosed an mRNA-based nanotherapeutic that produces the decoy hsACE2 protein to potentially inhibit the SARS-COV-2 infection. A potent LNP formulation (eLNP) herein disclosed is shown to deliver IVT mRNA to the cytosol, where it is translated into hsACE2 protein more efficiently than the conventional LNPs (LNPs containing cholesterol but lacking β-sitosterol). It is herein disclosed that hsACE2 protein that was generated from the LNP-delivered mRNA efficiently binds to the RBD of SARS-COV-2 with a high affinity. Additionally, hsACE2 exerts a potent neutralizing effect on the pseudovirus decorated with the S protein of SARS-COV-2.

Disclosed herein, intravenous injection of eLNP/hsACE2 is shown to enable rapid and sustained expression of the circulating hsACE2 protein in the blood circulation within 2 h, peaking at 6 h and clearing gradually. Lung transfection with eLNP/hsACE2 is shown to illicit secretion of hsACE2 protein to the airway mucus in which the primary infection of SARS-COV-2 occurs. Unlike Fc fragment fused chimeric hsACE2 protein, the availability of mRNA-derived circulating hsACE2 is due to continuous generation of new protein from the liver. This provides an opportunity for rapid clearance of the virus while providing protection against the dysregulated RAAS system due to long term presence of newly made protein in the serum.

Another use of recombinant hsACE2 as herein disclosed may be to regulate blood pressure in the Angiotensin II-dependent hypertension. The prevalence of hypertension among the elderly in the United States is more than 60%, and this age-group is also at high risk of COVID-19. In this regard, it is conceivable that expression of enzymatically active hsACE2 from the mRNA therapy could protect COVID-19 patients with hypertension from aggravation of cardiovascular diseases as well as viral infection.

Furthermore, sustained expression of hsACE2 during infection could facilitate ACE2-mediated lung protection, reduce the incidence of ARDS by neutralizing SARS-COV-2, and prevent RAAS dysregulation. Additionally, hsACE2 may bind SARS-CoV-2 in the bloodstream and reduce its ability to infect other peripheral organs. It is possible that, by binding and thus masking the RBD, hsACE2 may decrease the amplitude of inflammatory response that causes multiorgan failure.

Accordingly, in one aspect, embodiments herein provide for a method of treating a patient suffering from a condition or disease, comprising administering to the patient an effective amount of a therapeutic agent comprising one or more RNA molecules encapsulated by a lipid nanoparticle. The treating of the patient may reduce at least one or more signs or symptoms associated with the condition or disease.

In an example of the method, the RNA is mRNA, and encodes for a soluble form of human angiotensin-converting enzyme 2 (hACE2) and/or one or more variations thereof. The mRNA encoding the soluble form of hACE2 and/or one or more variations thereof may comprise one or more sequences of SEQ ID NOs: 1-13 as disclosed herein.

In examples, the condition or disease is a viral infection. In a particular example, the viral infection is caused by severe acute respiratory syndrome coronavirus 2 (SARS-COV-2).

In examples, the lipid nanoparticle is comprised of an ionizable lipid, a PEG lipid, β-sitosterol, and a structural lipid. In some examples, the lipid nanoparticle does not include cholesterol.

In examples, the therapeutic agent is administered to the patient intravenously. In some examples, the therapeutic agent is administered to the patient by inhalation.

In an example of the method, an expression of the soluble form of hACE2 and/or one or more variations thereof is dependent on a dosage of the therapeutic agent. In a particular example, the expression of the soluble form of hACE2 and/or one or more variations thereof is time-dependent with a highest level of expression around 6 hours after the administration of the therapeutic agent.

In another aspect, embodiments provide for a therapeutic agent for treating a patient suffering from a viral infection, comprising a lipid nanoparticle comprised of each of an ionizable lipid, a PEG lipid, a sterol and/or substitution for the sterol, and a structural lipid; and one or more mRNA molecules encoding at least a portion of a soluble protein encapsulated within the lipid nanoparticle.

Examples of ionizable lipids include 2,2-dilinoleyl-4-dimethylaminoethyl-[1,3]-dioxolane (DLin-KC2-DMA), (6Z,9Z,28Z,31Z)-Heptatriaconta-6,9,28,31-tetraen-19-yl 4-(dimethylamino)butanoate (DLin-MC3-DMA), 1,1′-((2-(4-(2-((2-(bis(2-hydroxydodecyl) amino)ethyl)(2-hydroxydodecyl)amino)ethyl)piperazin-1-yl) ethyl)azanediyl)bis(dodecan-2-ol) (C12-200), 3,6-bis(4-(bis(2-hydroxydodecyl) amino)butyl)piperazine-2,5-dione (cKK-E12), di((Z)-non-2-en-1-yl) 9-((4-(dimethylamino)butanoyl)oxy)heptadecanedioate (L319), (6Z,9Z,28Z,31Z)-19-(4-(dimethylamino) butyl)heptatriaconta-6,9,28,31-tetraen-19-ol (YSK12-C4), 1-methyl-4,4-bis (((9Z,12Z)-octadeca-9,12-dien-1-yl)oxy)piperidine (YSK05), 7-(4-(dipropylamino) butyl)-7-hydroxytridecane-1,13-diyl dioleate (CL4H6), heptadecan-9-yl 8-((2-hydroxyethyl)(6-oxo-6-(undecyloxy)hexyl)amino)octanoate (SM-102; Lipid 8), heptadecan-9-yl 8-((2-hydroxyethyl)(4-(nonyloxy)-4-oxobutyl)amino)octanoate (Lipid 9), heptadecan-9-yl 8-((2-hydroxyethyl)(8-(nonyloxy)-8-oxooctyl)amino)octanoate (Lipid 5), 6-[6-(2-hexyldecanoyloxy)hexyl-(4-hydroxybutyl)amino]hexyl 2-hexyldecanoate (ALC-0315), 1,2-dioleoyl-3-trimethylammonium-propane (DOTAP), and 1,2-dioleoyl-3-dimethylammonium-propane (DODAP), 1,2-dioleyloxy-3-dimethylaminopropane (DODMA), N1, N3,N5-tris(3-(didodecylamino)-propyl)benzene-1,3,5-tricarboxamide (TT3), 3-((4,4-bis(octyloxy)butanoyl)oxy)-2-((((3-(diethylamino) propoxy)carbonyl)oxy)methyl)propyl (9E, 12E)-octadeca-9,12-dienoate (LP01), 2-(di((9E,12E)-octadeca-9,12-dien-1-yl)amino)ethyl 3-(4-methylpiperazin-1-yl)propanoate (Lipid 10).

Examples of PEG lipids include 1,2-dimyristoyl-rac-glycero-3-methoxypolyethylene glycol (DMG-PEG), 1,2-distearoyl-rac-glycero-3-methylpolyoxyethylene (DSG-PEG), 1,2-dipalmitoyl-rac-glycero-3-methylpolyoxyethylene (DPG-PEG), N-(Methylpolyoxyethylene oxycarbonyl)-1, 2-distearoyl-sn-glycero-3-phosphoethanolamine (DSPE-PEG), N-(Methylpolyoxyethylene oxycarbonyl)-1,2-dipalmitoyl-sn-glycero-3-phosphoethanolamine (DPPE-PEG), N-(Methylpolyoxyethylene oxycarbonyl)-1,2-dimyristoyl-sn-glycero-3-phosphoethanolamine (DMPE-PEG), 1,2-dimyristoyl-sn-glycero-3-phosphoethanolamine-N-[methoxy (polyethylene glycol)-2000] (14:0 PEG), 2-[(polyethylene glycol)-2000]-N,N-ditetradecylacetamide (ALC-0159).

Examples of structural lipids include 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dipalmitoyl-sn-glycero-3-phosphocholine (DPPC), 1,2-dipalmitoyl-sn-glycero-3-phosphoethanolamine (DPPE), 1,2-dimyristoyl-sn-glycero-3-phosphoethanolamine (DMPE), 1,2-dioleoyl-sn-glycero-3-phospho-(1′-rac-glycerol) (DOPG).

Examples of sterols and/or substitutions for sterols include cholesterol, β-sitosterol, fucosterol, campesterol, stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N-(N′N′-dimethylaminoethyl) carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof, e.g., sodium cholate.

In an example, the lipid nanoparticle does not include cholesterol.

In some examples, the mRNA encodes for a soluble form of human angiotensin-converting enzyme 2 (hACE2) and/or one or more variations thereof. The mRNA encoding the soluble form of hACE2 and/or one or more variations thereof may comprise one or more sequences of SEQ ID NOs: 1-13 as disclosed herein.

In some examples, the viral infection is caused by severe acute respiratory syndrome coronavirus 2 (SARS-COV-2).

In some examples, the soluble form of hACE2 and/or one or more variations thereof may bind to a receptor-binding domain of a spike protein of the virus with a high affinity.

In some examples, the soluble form of hACE2 and/or one or more variations thereof may reduce the viral infection through competitive inhibition.

In another aspect, embodiments provide for a method of treating a patient suffering from an infection caused by severe acute respiratory syndrome coronavirus 2 (SARS-COV-2), comprising administering to the patient an effective amount of a therapeutic agent comprising one or more mRNA molecules encoding at least a portion of a soluble form of human angiotensin-converting enzyme 2 (hACE2) encapsulated by a lipid nanoparticle, the lipid nanoparticle including an ionizable lipid, a PEG lipid, β-sitosterol, and a structural lipid. The treating of the patient may reduce at least one or more signs or symptoms associated with the infection.

In some examples, the therapeutic agent is administered to the patient intravenously in a single dose or in multiple doses.

In some examples, the therapeutic agent is administered to the patient by inhalation in a single dose or in multiple doses.

In examples, the one or more mRNA molecules encoding the soluble form of hACE2 may comprise one or more sequences of SEQ ID NOs: 1-13 as disclosed herein.

In examples, the soluble form of hACE2 may bind to a receptor-binding domain of a spike protein of SARS-COV-2 and reduce the one or more signs or symptoms associated with the infection through a competitive inhibition of SARS-COV-2.

While in examples a LNP is used as a delivery vector, it is within the scope of this disclosure that additionally or alternatively other/another delivery vector may be used (e.g., lentiviral vector, plasmid expression vector, and the like).

### EXAMPLES

### Example 1

### Methods and Materials

### Materials

Fluc mRNA and hsACE2 variant mRNA were purchased from TriLink Biotechnologies (CA, USA). Uridine of Fluc mRNA was fully substituted with 5-methoxyuridine, and uridine and cytidine of hsACE2 mRNA were fully substituted with pseudouridine and 5-methyl-cytidine, respectively. Cholesterol and β-sitosterol were purchased from Sigma-Aldrich. DMG-PEG_{2K} was bought from NOF America. DLin-MC3-DMA and DSPC were obtained from BioFine International Inc. and Avanti Polar Lipids, Inc., respectively.

### LNP Formulation and Characterization

LNPs composed of DLin-MC3-DMA, Cholesterol or β-sitosterol, DMG-PEG_{2K}, DSPC, and mRNA were prepared using microfluidic mixing. Briefly, mRNA was diluted in sterile 50 mM citrate buffer, and lipid components were prepared in 100% ethanol at 50:38.5:1.5:10 molar ratio. The lipid and mRNA solutions were mixed using the NanoAssemblr Benchtop at a 1:3 ratio, followed by overnight dialysis against sterile PBS using a Slide-A-Lyzer G2 cassette with 10,000 Da molecular-weight-cut-off (Thermo Fisher Scientific). Dialyzed LNP solutions were concentrated using Amicon® Ultra centrifugal filter units with 10,000 Da molecular-weight-cut-off (Millipore). Hydrodynamic size and PDI of the LNPs were measured in dynamic light scattering using the Zetasizer Nano ZSP (Malvern Instruments, UK). mRNA encapsulation was assayed using a Quant-iT™ RiboGreen® RNA Assay kit (Thermo Fisher Scientific) and a multimode microplate reader (Tecan Trading AG, Switzerland).

### Cell Culture

293T, Calu-3, Hep G2 cell lines were kindly gifted from Prof. Sadik Esener (OHSU), Prof. Kelvin MacDonald (OHSU), and Prof. Conroy Sun (OSU), respectively. 293T/17 cell line was purchased from ATCC (CRL-11268). 293T, 293T/17 and Hep G2 cells were cultured in DMEM supplemented with 10% heat-inactivated FBS and 1% penicillin/streptomycin. Calu-3 cells were cultured in MEM supplemented with 10% heat-inactivated FBS, 1% penicillin/streptomycin, non-essential amino acids, and sodium pyruvate.

In Vitro Fluc mRNA Transfection Assay

For in vitro Fluc mRNA transfection assays, cells were seeded on a white 96 well plate at 4×10³ cells/well for 293T and Hep G2 cells or at 10⁴ cells/well for Calu-3, followed by overnight incubation for cell attachment. Cells were incubated with nanoparticles encapsulating Fluc mRNA and analyzed for cell viability and luciferase activity with the ONE-Glo™+Tox luciferase reporter and cell viability assay kit (Promega) using a multimode microplate reader.

In Vitro hsACE2 mRNA Transfection

For in vitro mRNA transfection for hsACE2 production, cells were seeded on a 12-well plate at 3×10⁵ cells per well and allowed to attach for overnight. Cells were treated with LNPs encapsulating hsACE2 mRNA for 24 h, and culture media were centrifuged at 500 g for 10 min at 4° C. Cell-free media was supplemented with protease and phosphatase inhibitor cocktail (Thermo Fisher Scientific) and used for downstream experiments. Besides culture media, transfected cells were lysed using RIPA buffer containing protease and phosphatase inhibitor cocktail, followed by centrifugation at 16,000 g for 30 min at 4° C. Supernatant lysate was collected for western blot.

Detection of hsACE2 Protein by Western Blot

Production of hsACE2 protein upon transfection was detected by western blot. In brief, total protein concentration of sample was quantified using a Micro BCA protein assay kit (Thermo Fisher Scientific) according to the manufacturer's instruction. Cell-free supernatants or cell lysates containing 30 μg of total protein were prepared in 1× LDS sample buffer under reducing conditions, denatured at 70° C. for 10 min, and run on 4-12% Bis-Tris gels or 4-20% Tris-glycine gels, followed by dry transfer to PVDF membrane using iBlot 2 Dry Blotting System (Thermo Fisher Scientific). The blots were blocked using 5% skim milk for 1 h at room temperature. The primary antibodies used were: rabbit monoclonal anti-V5 tag at 1:1,000 (Cell Signaling Technology, 13202), rabbit monoclonal anti-6x-His tag at 1:1,000 (Thermo Fisher Scientific, MA5-33032), and mouse monoclonal anti-β-actin at 1:10,000 (R&D Systems, MAB8929). The secondary antibodies used were goat polyclonal anti-rabbit HRP (Jackson ImmunoResearch, 111-035-003) and anti-mouse HRP (115-035-003). For detection and documentation, we used SuperSignal™ West Pico Plus Chemiluminescent Substrate and myECL imager (Thermo Fisher Scientific). After chemiluminescent imaging, blots were further stained using GelCode™ Blue Safe Protein Stain (Thermo Fisher Scientific) according to the manufacturer's instruction.

Co-Immunoprecipitation of hsACE2 and SARS-COV-2 Spike RBD

Cell free media from untreated or transfected 293T cell culture was prepared. 1 μg of SARS-COV-2 Spike RBD-His (Sino Biological) was inoculated to 400 μl of cell free media, followed by overnight incubation at 4° C. with rotation. Subsequent co-immunoprecipitation was conducted using Dynabeads™ Protein G Immunoprecipitation kit (Thermo Fisher Scientific) according to the manufacturer's instruction. Briefly, cell-free media inoculated with the spike RBD were incubated with antibody bound Dynabeads for 20 min at room temperature with rotation. The antibodies used for pull-down were mouse monoclonal anti-His tag (sc-8036) or anti-V5 tag (sc-81594) antibody (Santa Cruz Biotechnology). Following three washes with PBS, samples were eluted using elution buffer and denatured using LDS sample buffer and reducing agent for western blot.

### Animals

All animal studies were conducted at Oregon Health and Sciences University and approved by the Institutional Animal Care and Use Committee (IACUC, IP00001707).

In Vivo Fluc mRNA Transfection via Intravenous Administration

Female BALB/c mice (8-12 weeks) were sedated using isoflurane, and LNPs encapsulating Fluc mRNA were intravenously administered via tail vein. At predetermined time points post-administration, 200 μl of D-luciferin substrate was intraperitoneally injected to the mice 10 minutes prior to bioluminescence imaging (150 mg/kg). Image acquisition and analysis were performed using the IVIS® Lumina XRMS and the manufacturer's software (PerkinElmer).

In vivo hsACE2 mRNA Transfection via Intravenous Administration

Female BALB/c mice (8-12 weeks) were sedated using isoflurane, and LNPs encapsulating hsACE2 mRNA were administered to animals via tail vein. At predetermined time points post-administration, whole blood was collected using cardiac puncture or submandibular bleeding. The collected blood samples were processed to sera using serum-separating tubes (BD). The separated sera were used for downstream experiments. Mouse liver were sterilely harvested and homogenized using a handheld tissue homogenizer.

In vivo mRNA transfection via Intratracheal Instillation

Intratracheal instillation was performed according to established protocols. Female BALB/c mice (8-12 weeks) were anesthetized using ketamine/xylazine cocktail. Anesthetized animals were leaned over intubation stand (Kent Scientific), and their vocal cords were directly visualized using an otoscope with a 2-mm speculum (Welch Allyn). A flexible guide wire was advanced through the vocal cords to trachea. Once the wire was located within trachea, a 20 G catheter was passed over the wire and the wire was removed. To administer LNPs, a gas tight syringe with a 22 G blunt needle (Hamilton) was filled with LNPs containing mRNA. The syringe were inserted through the catheter and LNPs encapsulating mRNA was administered to lungs, followed by 100 μl of air to distribute the LNP solution throughout the lungs.

### Collection of Bronchoalveolar Lavage Fluid (BALF)

After intratracheal instillation, euthanize the animals by CO₂ asphyxia at an appropriate time post-administration. The trachea was surgically exposed and intubated with a 20 G catheter. The mouse lungs lavage was performed three times with 0.8 ml of prewarm PBS to collect BALF. The collected BALF was centrifuged at 500 g for 10 min at 4° C. The supernatants were supplemented with protease and phosphatase inhibitor cocktail and used for downstream experiments.

Immunoprecipitation of hsACE2 from BALF

Immunoprecipitation of hsACE2 from BALF was conducted using Dynabeads™ Protein G Immunoprecipitation kit (Thermo Fisher Scientific) according to the manufacturer's instruction. The collected BALF was incubated with Dynabeads having anti-V5 tag antibody for 20 min at room temperature with rotation. Following three washes with PBS, samples were eluted using elution buffer and denatured using LDS sample buffer and reducing agent for western blot.

### Plasmids

Lentiviral reporter plasmid pHAGE-CMV-Luc2-IRES-ZsGreen-W (BEI Resources, NR-52516) and helper plasmids pHDM-Hgpm2 (BEI Resources, NR-52517), pHDM-tatb (BEI Resources, NR-52518), pRC-CMV-rev1b (BEI Resources, NR-52519), and hACE2 containing pHAGE2-EF1a ACE2 (BEI Resources, NR-52512) were kindly provided by Jesse D. Bloom. pcDNA3.1-SARS2-Spike (Addgene, #145032) was a gift from Fang Li. pMD2.G containing VSV-G envelope protein (Addgene, #12259) and pCMVΔR8.2 (Addgene, #12263) were a gift from Didier Trono.

### Generation of 293T-hACE2

In order to create 293T/17 cells overexpressing hACE2, we transduced the hACE2 gene to 293T/17 cells using a lentiviral vector. To produce the lentivirus packaging hACE2 gene, 293T/17 cells were transfected with pCMVΔR8.2, pMD2.G, and pHAGE2-EF1aInt-ACE2-WT using lipofectamine 2000. After 4 h, the cells were replenished with the fresh growth media. After 48 h, the lentiviral particles were collected, filtered, and used immediately to transduce 293T/17 cells. After 48 h transduction, the cells were harvested, passaged with the growth media, and referred to as 293T-hACE2. To confirm the expression of hACE2 after transduction, 293T-hACE2 cells were harvested and lysed using RIPA buffer containing protease and phosphatase inhibitor cocktail. Cell lysates was processed to perform western blot analysis as described above. To probe hACE2, anti-ACE2 antibody (Santa Cruz Biotechnology, sc-390851) and anti-mouse HRP were used as the primary and secondary antibodies at 1:200 and 1:2,000, respectively.

### Production of Pseudovirus Particles

293T/17 cells were seeded in T-75 flask at 5×10⁴ cells/flask and grown for 18 h. Cells were co-transfected with 7.8 μg of the lentiviral reporter, 1.7 μg of each helper plasmids, and either 7.8 μg of pcDNA3.1-SARS2-Spike (Spike), 2.5 μg of pMD2.G (VSV-G), or no plasmid (No envelope) using lipofectamine 3000 as instructed by manufacture. After 48 h, pseudoviruses were collected, filtered, aliquoted into single-use vials, and stored at −80° C.

### Titration of Pseudovirus Particles

293T-hACE2 cells were seeded at 10⁴ cells/well in white, 96-well plates and grown for 18 h. Cells were transduced in triplicate with a 4-point, 1:3 serial dilution of the pseudoviruses with polybrene at a final concentration of 5 μg/ml. Polybrene was not included in the VSV-G pseudovirus-treated wells. After 48 h, cell viability and luciferase activity were assessed with the ONE-Glo™M+Tox luciferase reporter and cell viability assay kit.

### Preparation of Conditioned Media for Pseudovirus Neutralization Assay

To make conditioned media containing hsACE2, 293T/17 cells were seeded into T-75 flasks at 5×10⁴ cells/flask and grown 18 h. Cells were transfected with 22 μg mRNA or equivalent volume of PBS using lipofectamine 3000. After incubation for 6 h, cells were washed with PBS and the complete media was added. After 24 h, media was harvested, filtered with 0.45 um filter, and concentrated in a spin column with Amicon® Ultra centrifugal filter units with 10,000 Da molecular-weight-cut-off at 4,000 g for 30 minutes. The concentrated, conditioned media was brought up to 2 ml with serum-free media and used immediately in the neutralization assay.

### Pseudovirus Neutralization Assay

For neutralization assay, 293T-hACE2 cells were seeded into white 96-well plates at 2×10⁴ cells/well and grown for 24 h. Pseudovirus was serially diluted as before. The conditioned media was added to the serial dilutions at ratio of 2:3 for conditioned media: pseudovirus, and incubated at 4° C. for 1 h. Polybrene was added as before. Media was removed from the 96-well plates and cells were transduced as before. After 48 h, cell viability and luciferase activity were assessed with the ONE-Glo™M+Tox luciferase reporter and cell viability assay kit.

### Example 2

Design of mRNA-based Nanotherapeutic to Treat SARS-COV-2 Infection

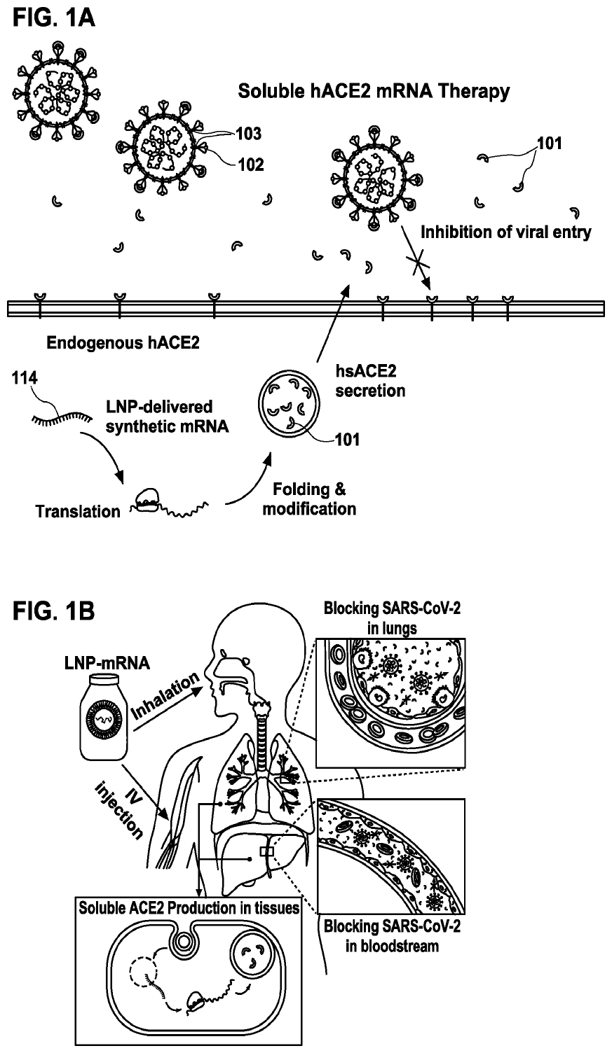

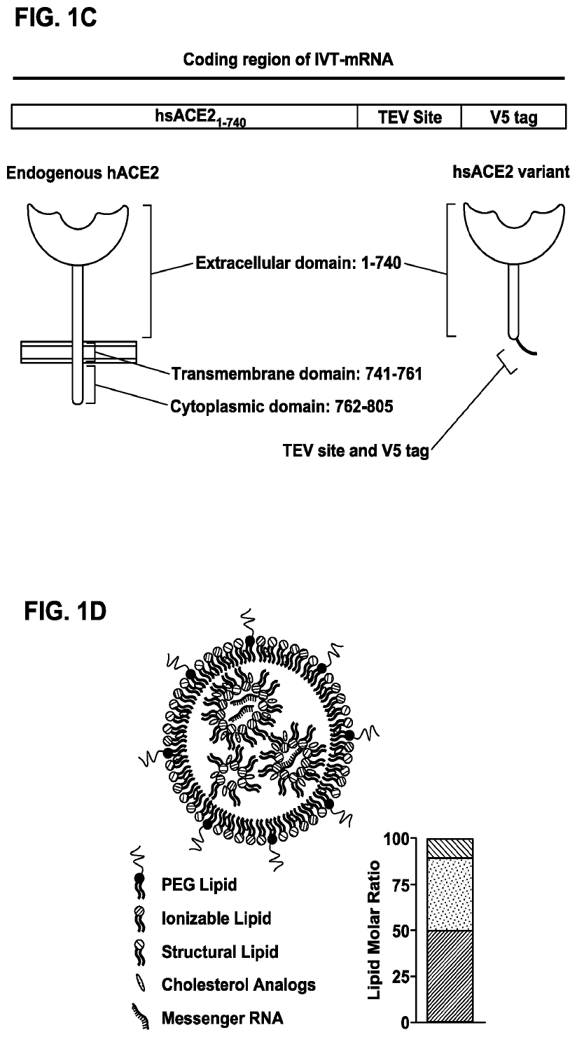

LNPs were used to deliver in-vitro-transcribed messenger RNA (IVT mRNA) for rapid expression of hsACE2. FIG. 1A illustratively depicts a rationale for soluble ACE2 (hsACE2) messenger RNA (mRNA) therapeutics in treating SARS-COV-2 infection. Briefly, LNP-delivered synthetic mRNA 114 generates human soluble ACE2 (hsACE2) protein 101 that is secreted into the extracellular compartment where it binds receptor binding domain 102 of the spike protein 103 of the SARS-COV-2 and prevents viral entry. FIG. 1B illustrates potential routes of administration for soluble ACE2 mRNA therapeutic. Intravenous administration of the LNPs encapsulating hsACE2 mRNA leads to the production of circulating hsACE2 protein from the liver. Inhalation of the LNPs leads to production of mucosal hsACE2 protein in the lungs. FIG. 1C depicts a schematic of in-vitro-transcribed (IVT) mRNA encoding hsACE2 variant protein, TEV site, and V5 tag (top). Endogenous hACE2 consists of three domains: extracellular, transmembrane, and cytoplasmic domains (bottom left). The hsACE2 variant lacks transmembrane and cytoplasmic domains of the endogenous hACE2 protein, and it contains TEV site and V5-epitope tag at C-terminus (bottom right). FIG. 1D depicts a schematic representation of LNP encapsulating mRNA. Ionizable lipid 105, cholesterol analogs 108, structural lipid 110, PEG lipid 112, mRNA 114.

### Example 3

Characterization of hsACE2 Expression

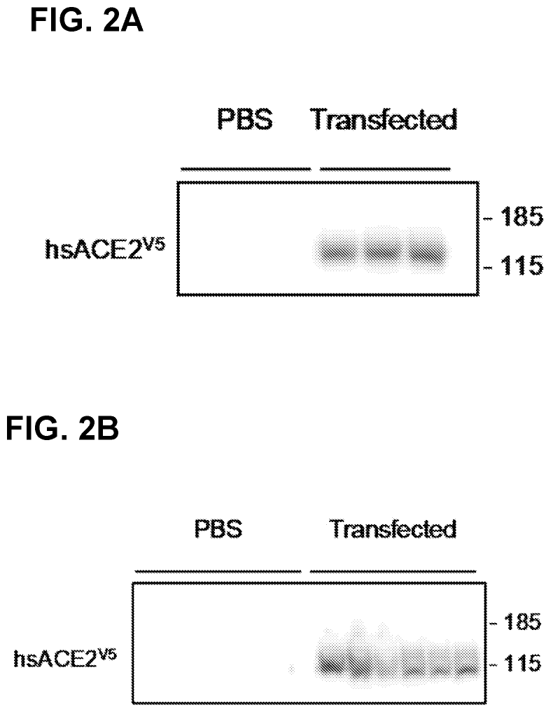

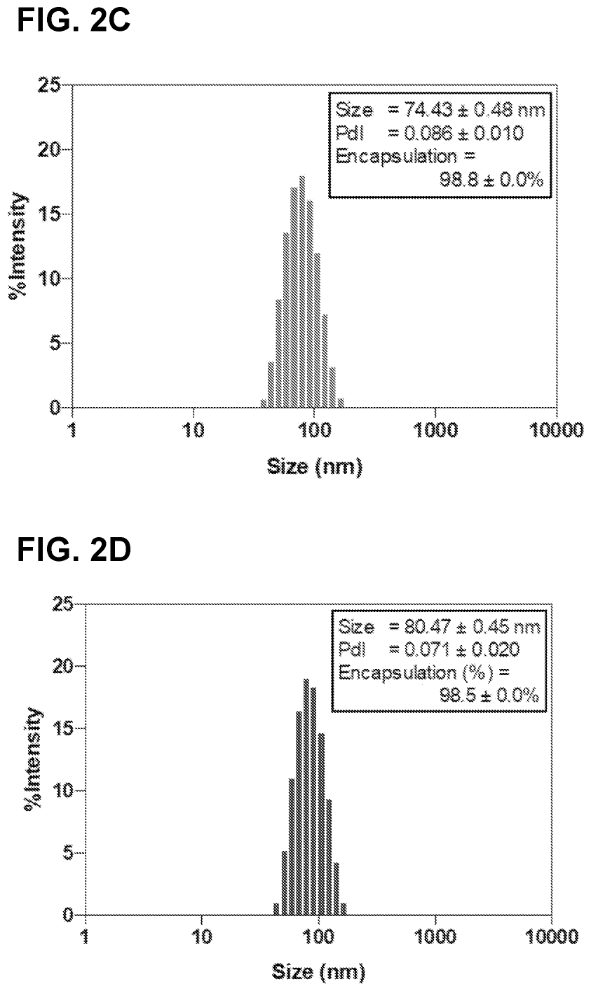

To confirm whether the designed IVT mRNA produces hsACE2 protein after transfection, 293T cells were transfected with hsACE2 mRNA using lipofectamine 3000. hsACE2 protein was detected in cell-free conditioned media (FIG. 2A) and cell lysates from hsACE2 mRNA transfected cells (FIG. 2B) by western blot, but not in PBS-treated controls (FIG. 2A-2B).

Intracellular delivery of mRNA, especially in vivo, may be more efficient with a delivery vector (e.g., LNP). LNPs may be comprised of at least four lipids: (1) ionizable lipid, (2) PEG lipid, (3) cholesterol, and (4) structural lipid (FIG. 1D). Substitution of cholesterol to β-sitosterol within LNP formulations may boost intracellular delivery of mRNA. An enhanced LNP formulation (eLNP: containing β-sitosterol) was compared with the LNPs lacking β-sitosterol (containing cholesterol) as the delivery vector for hsACE2 mRNA. Both LNP and eLNP encapsulating hsACE2 mRNA (LNP or eLNP/hsACE2) exhibited comparable characteristics in terms of hydrodynamic sizes (≈80 nm), polydispersity (PdI≈0.08), and RNA encapsulation (above 98%). FIGS. 2C-2D show representative data of size distribution and RNA encapsulation of LNP/hsACE2 (FIG. 2C) and eLNP/hsACE2 (FIG. 2D) used in the present disclosure.

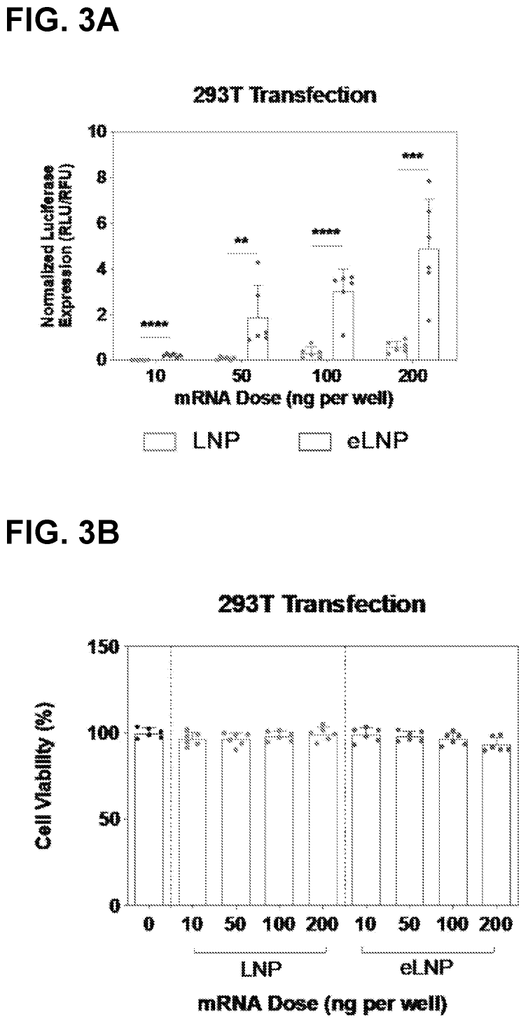

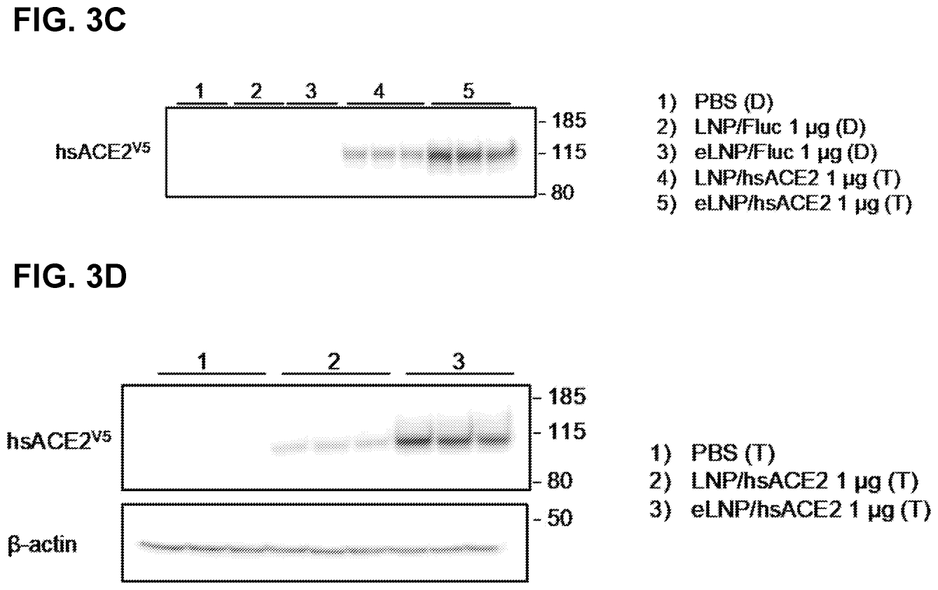

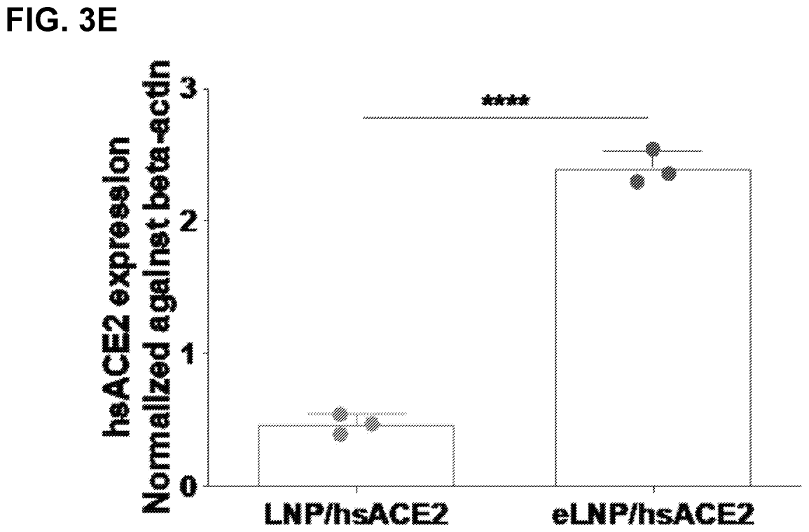

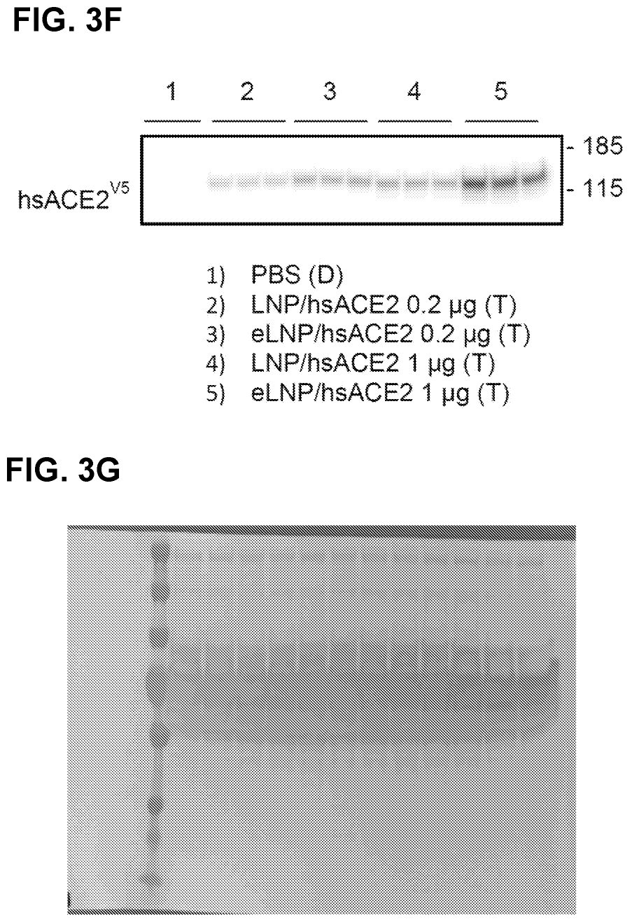

It was found that eLNP encapsulating firefly luciferase (Fluc) mRNA generated significantly higher luciferase expressions than LNP (p<0.01) with a dose-dependent manner in 293T cells without decreasing cell viability, indicating improved transfection efficiency (FIGS. 3A-3B). At FIG. 3A, the order is LNP (left), followed by eLNP (right) for each mRNA dose. In hsACE2 mRNA delivery, eLNP/hsACE2 elicited substantially greater production of hsACE2 protein compared to LNP/hsACE2 in cell-free conditioned media from 293T culture. In the cell-free conditioned media from 293T cells treated with LNP/Fluc and eLNP/Fluc, no expression of hsACE2 was detected in western blot (FIG. 3C). The improved hsACE2 expression by eLNP was confirmed again in the analysis of cell lysates (5-fold higher expression, p<0.0001) (FIGS. 3D-3E). Additionally, the production of hsACE2 protein was dependent on the mRNA dose given (FIGS. 3F-3G). For FIGS. 3A-3B and FIG. 3E, statistical analysis was performed using Student's t test. ** p<0.01, *** p<0.001, **** p<0.0001. All data were expressed as the mean±S.D.

Invasion of SARS-COV-2 in ACE2-expressing airway epithelial cells maybe followed by infection of endothelial cells, which may thereby lead to endotheliitis. Vascular leakage caused by damaged endothelial cells may provide the virus with a putative gateway to the circulatory system and other ACE2-expressing organs.

Therefore, blockade of influx of the virus from the blood circulation to peripheral organs may prevent multisystem organ failure.

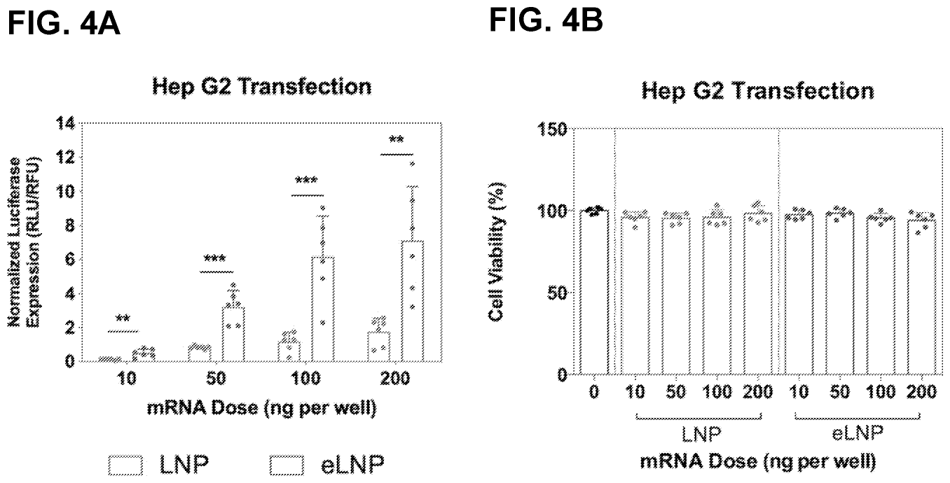

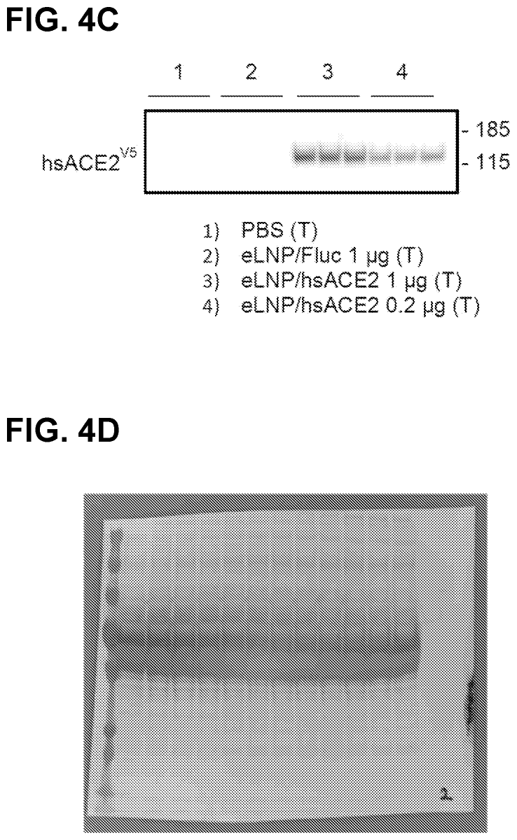

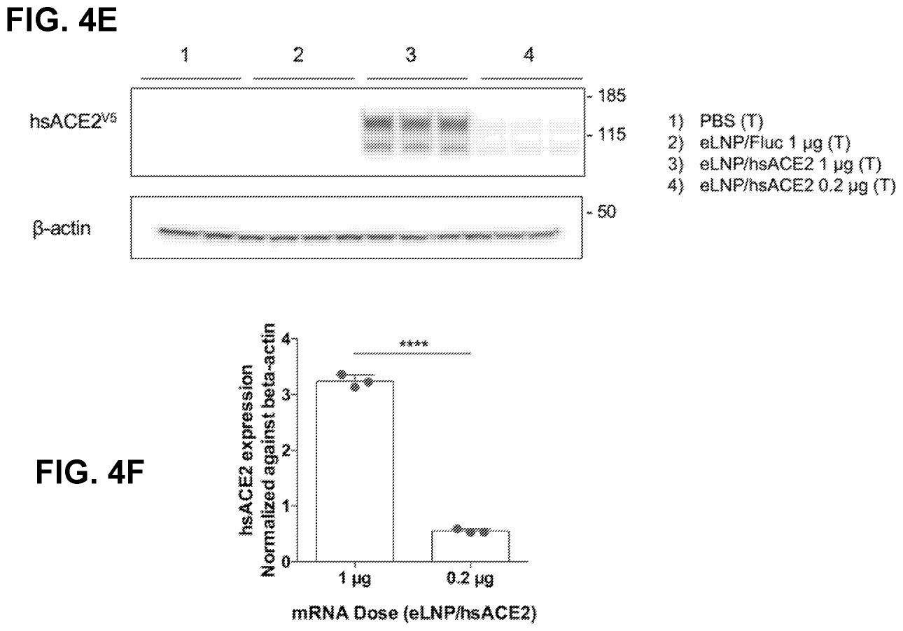

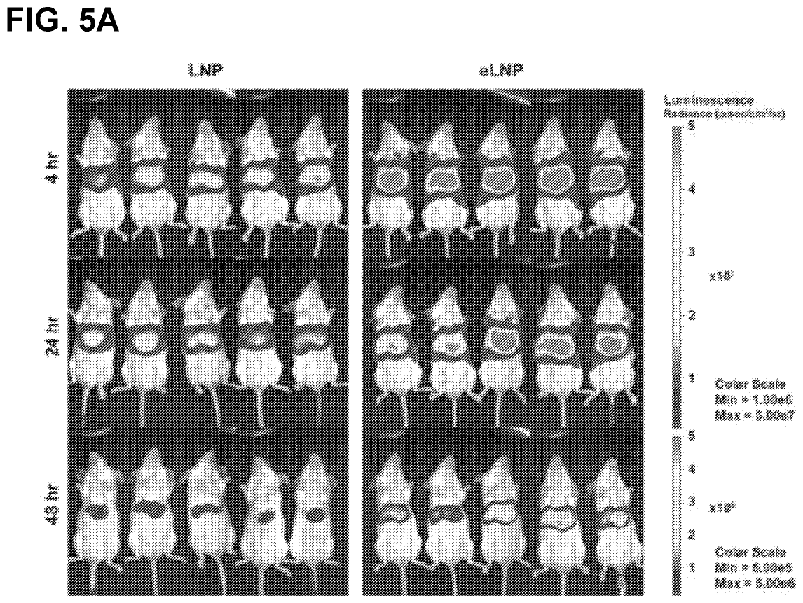

For these reasons, in vivo delivery of eLNP/hsACE2 was evaluated for production, secretion, and blood circulation of hsACE2 protein. Intravenously administered LNPs are typically destined to transfect hepatocytes owing to the interaction between LNP and apolipoprotein E. Thus, it was theorized that the liver may serve as a factory for protein production upon hsACE2 mRNA transfection. To assess this in vitro, the human liver cell line, Hep G2, was transfected with LNPs. eLNP/Fluc yielded more luciferase expression than LNP/Fluc in Hep G2 cells (FIG. 4A-4B). At FIG. 4A, the order is LNP (left), followed by eLNP (right) for each mRNA dose. As was the case with 293T cells, hsACE2 protein was found within the cell-free conditioned media and lysates that were harvested from the transfected Hep G2 cells in a dose-dependent manner (FIGS. 4C-4F). At FIGS. 4A-4B and FIG. 4F statistical analysis was performed using Student's t test. ** p<0.01, *** p<0.001, **** p<0.0001. All data were expressed as the mean±S.D.

In the Hep G2 cell lysates, two discrete hsACE2 bands were observed. We assume the band at approximately 125 kDa represented the fully-glycosylated form and the band at 100 kDa represented the pre- or partially-glycosylated form (FIG. 4E). However, only the fully-glycosylated form was detected in the cell-free media (FIG. 4C), suggesting the glycosylation of hsACE2 protein is prior to secretion.

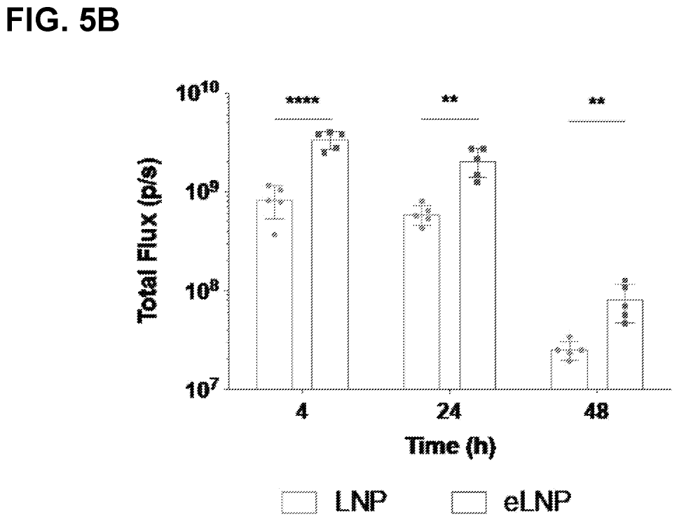

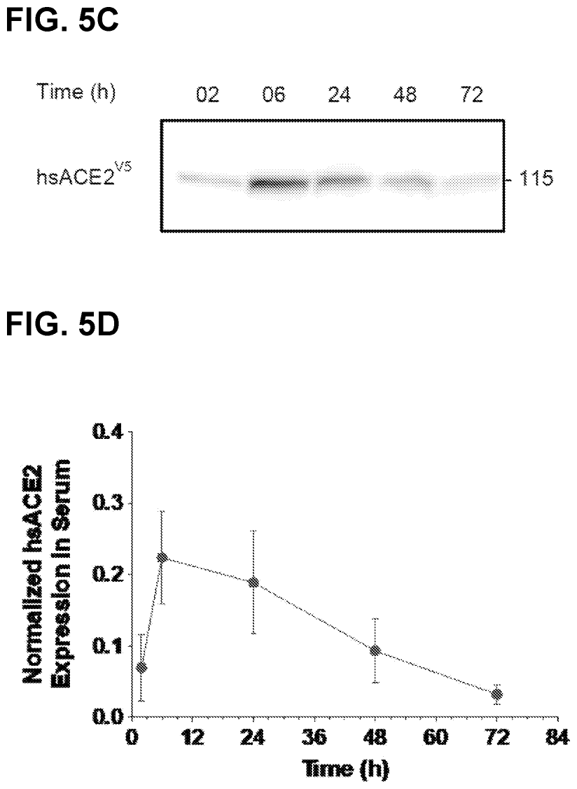

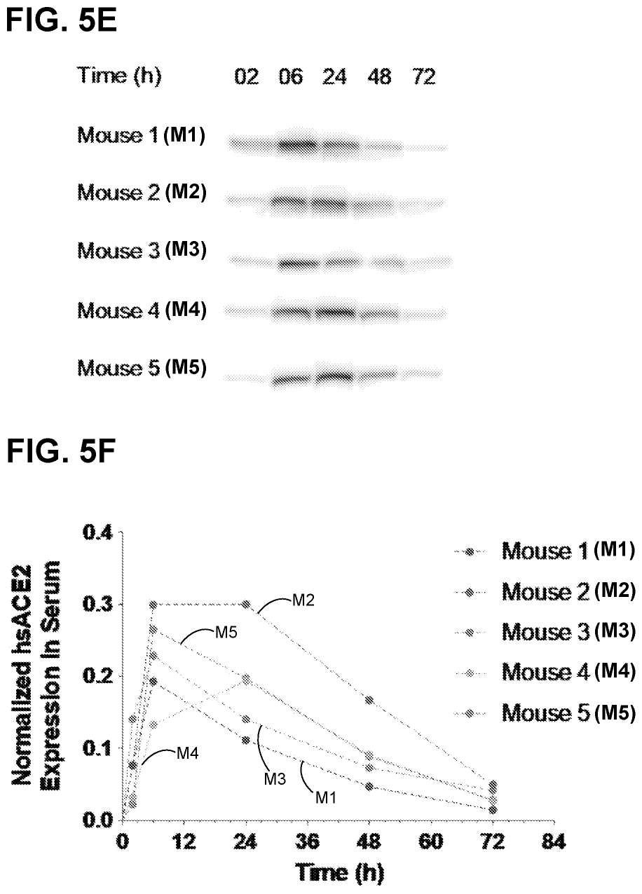

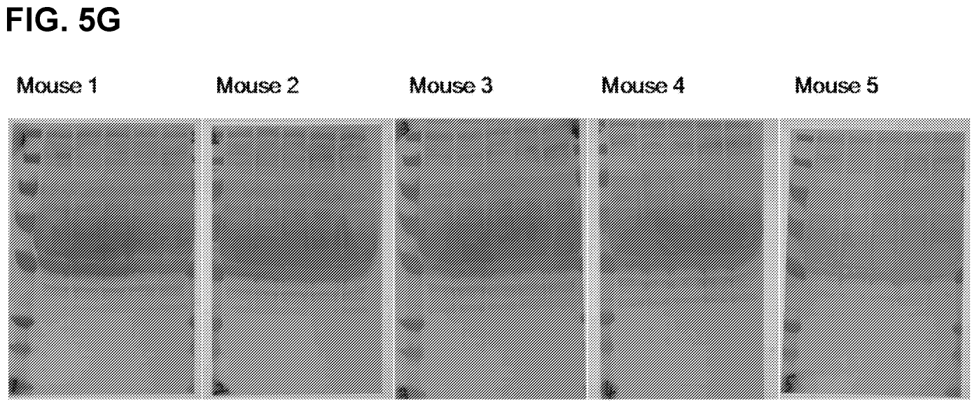

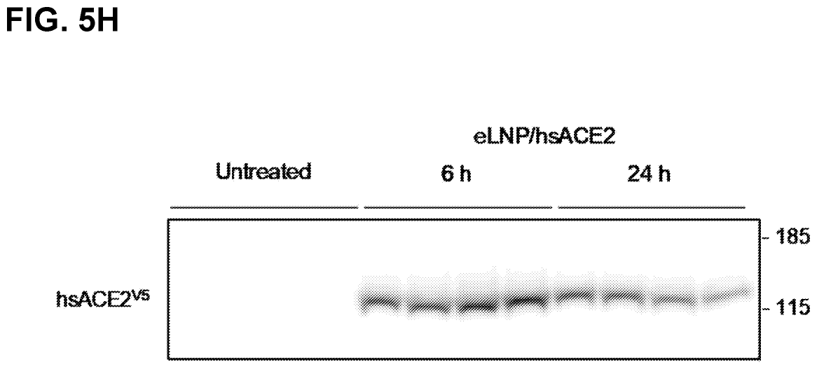

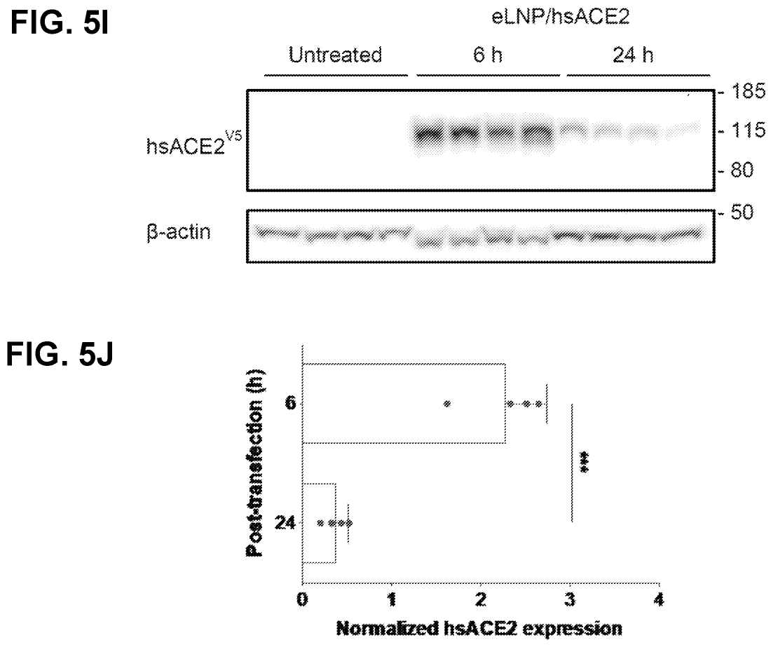

It was next examined whether the eLNPs lead to improved protein production in vivo. It was shown that eLNP/Fluc induced strong bioluminescent signals in the livers of BALB/c mice after intravenous injection of LNPs at 4 hours post-injection, which decreased with time (FIG. 5A). eLNP/Fluc exhibited 3-fold increase in luciferase expression as compared to LNP/Fluc at all time points (4-48 h) (FIG. 5B). Based on these results, eLNP was used as an optimized formulation for delivering mRNA in the remaining studies.

eLNP/hsACE2 was injected in BALB/c mice and mouse sera was collected up to 72 h post-administration with predetermined time intervals. Notably, hsACE2 appeared in the mouse sera as early as 2 h post-injection (FIGS. 5C-5G). The rapid generation of hsACE2 from the liver may be useful to neutralize SARS-COV-2 promptly at a stage of systemic spread. It was found that hsACE2 was detected at the highest level at 6 h post-injection and gradually declined afterwards. Circulating hsACE2 could be detected even 72 h after a single injection (FIGS. 5B-5C and FIG. 5H). The expression of hsACE2 protein in liver homogenates was time-dependent, showing a greater expression of the protein after 6 h than 24 h post-administration (p<0.001) (FIGS. 5I-5J). Unlike the cell lysates of Hep G2, the mouse liver homogenates showed a single band at approximately 125 kDa. After 7 days, hsACE2 was mostly eliminated from the blood circulation (data not shown).

### Example 3

LNP-Delivered hsACE2 mRNA to the Lungs Results in Production of Mucosal hsACE2 Protein

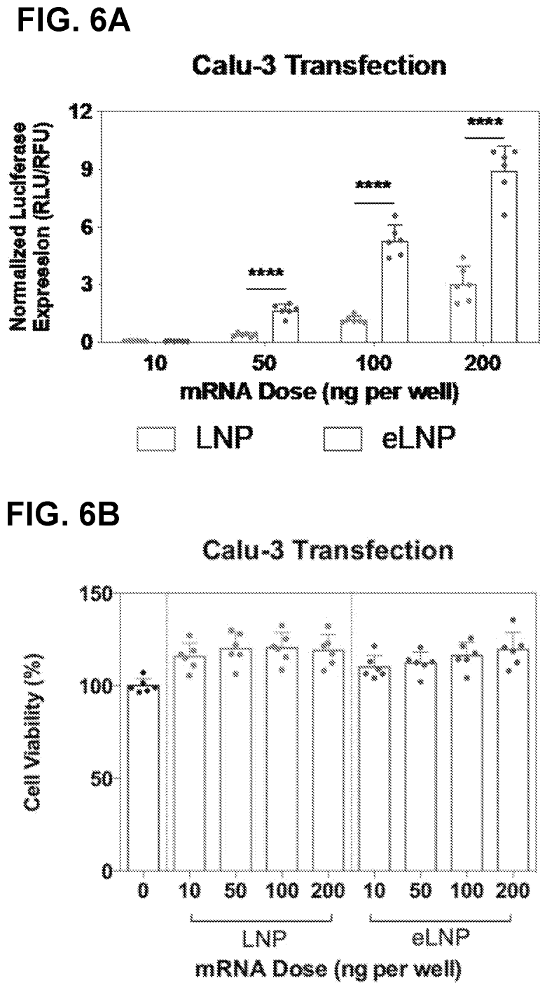

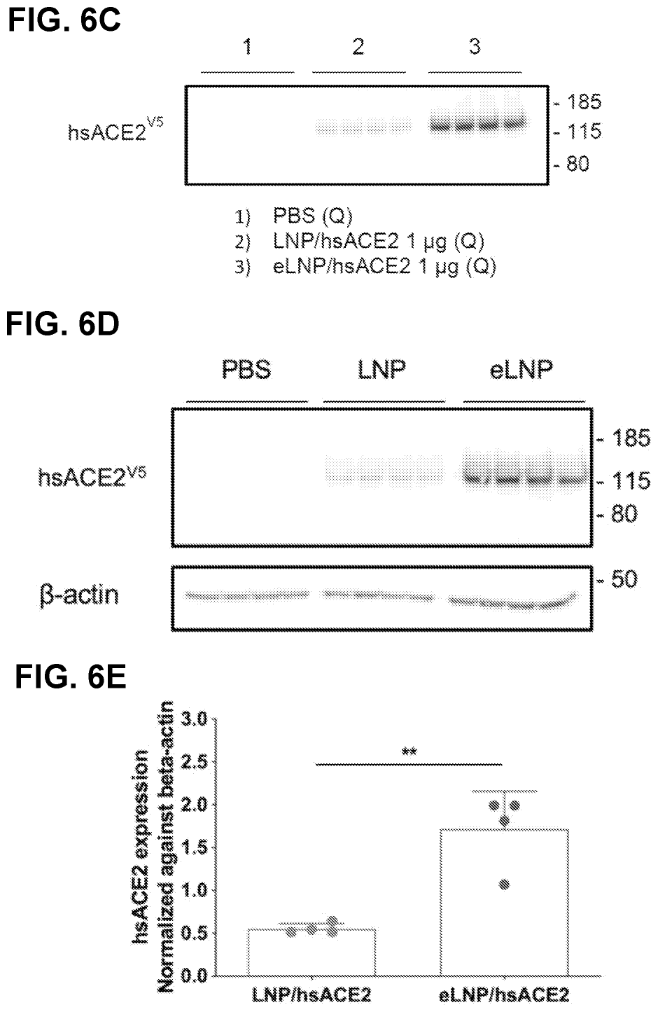

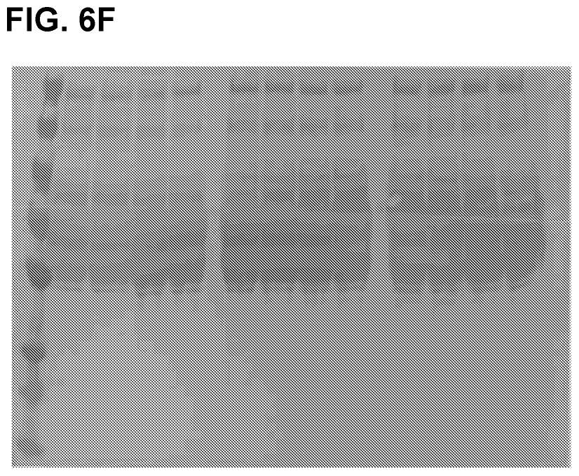

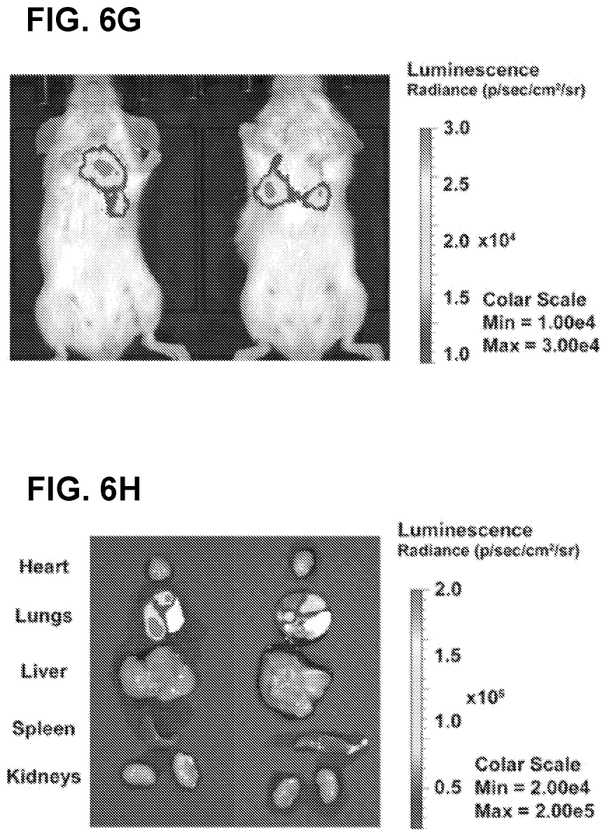

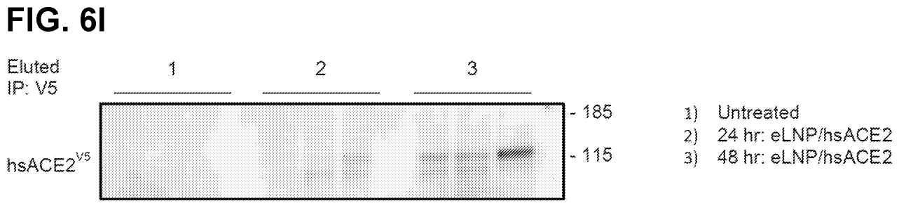

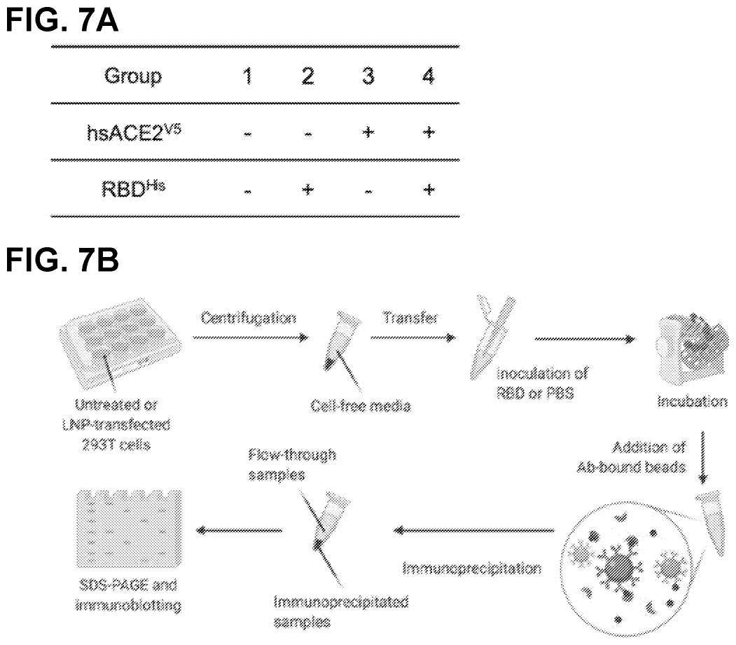

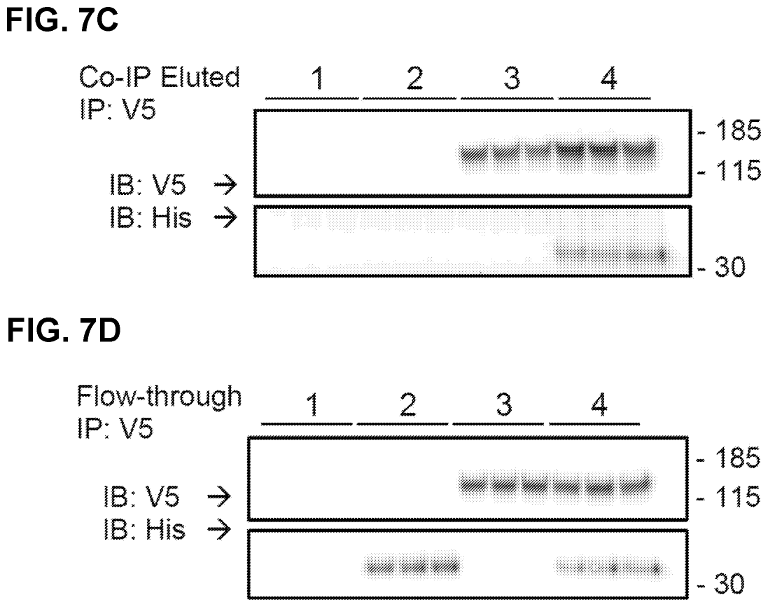

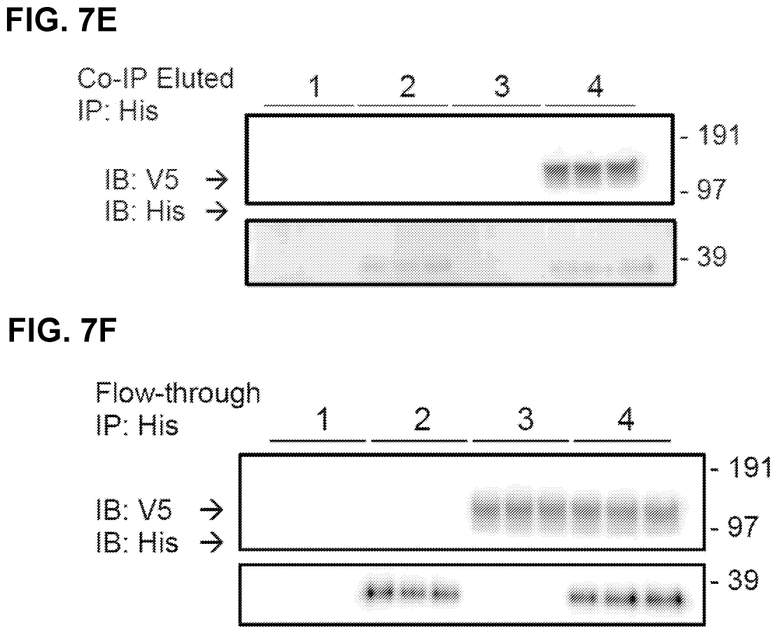

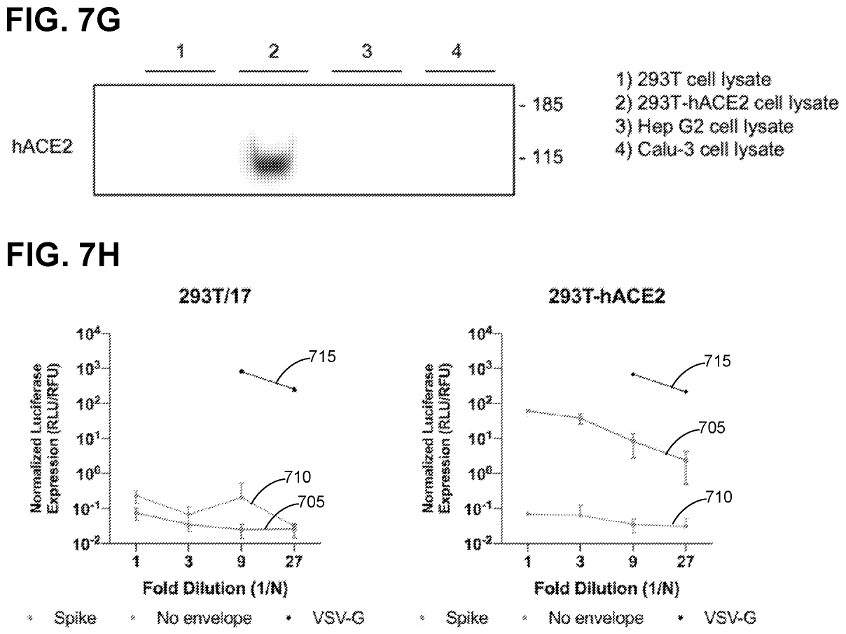

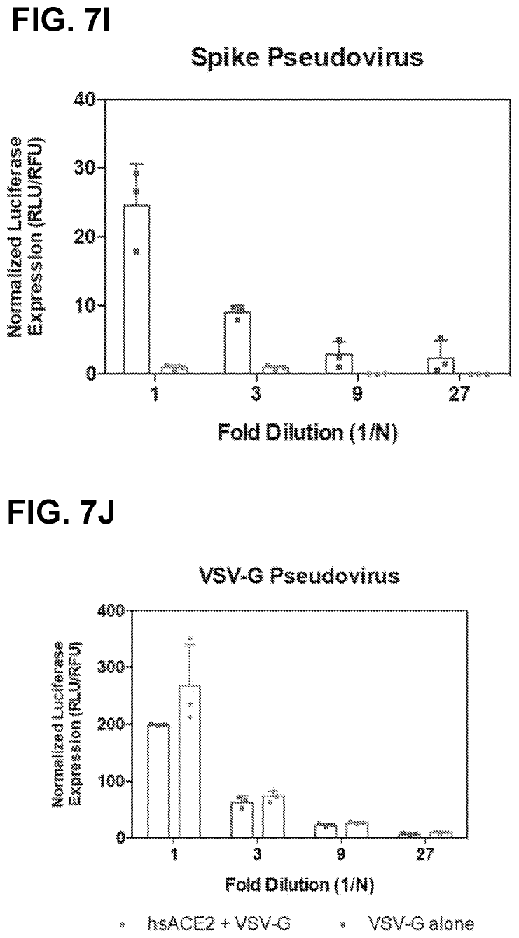

Airway and lungs are the first target organs where the virus attacks and are highly vulnerable organs due to high levels of hACE2 expression. Having hsACE2 protein as a decoy on the airway epithelium could mitigate viral infection at early stages of disease progression. Therefore, the ability of LNPs to produce mucosal hsACE2 was assessed. Consistent with the previous results, eLNP/Fluc exerted significantly greater levels of transfection than LNP/Fluc in Calu-3, a human lung epithelial cell line (FIGS. 6A-6B). Similarly, eLNP/hsACE2 showed substantially higher expression of hsACE2 protein than LNP/hsACE2 in western blot (FIGS. 6C-F). To locally deliver LNPs to the mouse lungs, we used intratracheal instillation as the route of administration. Intratracheally administered eLNP/Fluc transfected the lungs of BALB/c mouse, and the luciferase expression was detected exclusively in the lungs (FIGS. 6G-6H). To evaluate the secretion of hsACE2 to the lung mucosa, bronchoalveolar lavage fluid (BALF) samples were collected at 24 h and 48 h post-administration of eLNP/hACE2, followed by pull-down of hsACE2 protein using anti-V5 antibody. We confirmed the presence of mucosal hsACE2 in the collected BALF samples by western blot (FIG. 6I). These data represent that lung transfection with eLNP/hsACE2 resulted in the secretion of hsACE2 to the airway mucus.

### Example 4

hsACE2 Protein Binds the RBD and Prevents S1-Pseudovirus Infection

Next, it was evaluated whether there was a physical interaction of hsACE2 protein with the RBD of SARS-COV-2. 293T cells were transfected with eLNP/hsACE2 for 24 h, and untreated cells served as controls. Cell-free conditioned media was collected and inoculated with either PBS or the recombinant His-tagged RBD (FIGS. 7A-7B). Co-immunoprecipitation was performed with the samples using anti-V5 antibody to capture hsACE2^{V5} (FIGS. 7B-7C). SDS-PAGE was performed with the immunoprecipitated samples and the flow-through samples, followed by immunoblotting with anti-V5 and anti-His antibodies. It was observed that anti-V5 antibody was able to immunoprecipitate the hsACE2^{V5} from cell-free conditioned media (FIG. 7C) while the unbound hsACE2^{V5} was detected in the flow-through samples (FIG. 7D). The RBD^{His} was detected only in the immunoprecipitated samples that had both hsACE2 and the RBD (FIG. 7C) while the samples that contained RBD^{His} or hsACE2 alone showed no RBD^{His} band in the immunoprecipitated samples (FIG. 7C). The unbound RBD^{His} and hsACE2 were clearly detected in the flow-through samples (FIG. 7D). A reciprocal co-immunoprecipitation was also conducted, in which anti-His antibody was used to capture RBD^{His} (FIG. 7E-7F). It was observed that anti-His antibody co-immunoprecipitated hsACE2^{V5} in the samples that contained both hsACE2^{V5 a}nd RBD^{His}. hsACE2^{V5} band did not appear in the samples from other groups in immunoblotting. These results demonstrated that hsACE2 protein formed a protein complex with a specific and high-affinity association with the RBD of SARS-COV-2. The ability of hsACE2 to inhibit the transduction of the virus was further explored using a pseudovirus neutralization assay. To investigate hACE2-dependent infection of the pseudovirus, 293T cells stably expressing hACE2 (293T-hACE2) were created with a lentiviral vector. Expression of hACE2 in the transduced cells was examined in western blot, which showed hACE2 band at approximately 115 kDa (FIG. 7G). For pseudotyping the SARS-COV-2, Fluc-packaged HIV-based lentiviral particles containing the S protein of SARS-COV-2 on them were utilized. The lentiviral particles with vesicular stomatitis virus G protein (VSV-G) instead of the S protein were prepared as a positive control. It was found that the spike pseudovirus infection was hACE2-dependent; while VSV-G pseudovirus infection was not affected by hACE2 expression in host cells (FIG. 7H). After the hACE2 specificity of the spike pseudovirus was examined, the effects of hsACE2 on the pseudovirus infection was studied. To do this, hsACE2 conditioned media was produced by transfecting hsACE2 mRNA in 293T/17 cells. The pseudovirus and conditioned media containing hsACE2 were co-incubated on 293T-hACE2 cells and viral transduction was measured. It was found that cells treated with hsACE2 conditioned media led to a drastic reduction (more than 95%) of pseudovirus transduction as compared to the cells treated with pseudovirus alone (FIG. 7I). The VSV-G pseudovirus transduction was not affected by either treatment (FIG. 7J). These data highlight the potent inhibitory effect of hsACE2 treatment on the SARS-COV-2 infection by blocking the S protein-mediated viral attachment.

### Example 5

mRNA Sequence of the hsACE2 (SEQ ID NO: 1)

aug uca agc ucu ucc ugg cuc cuu cuc agc cuu guu
gcu gua acu gcu gcu cag ucc acc auu gag gaa cag
gcc aag aca uuu uug gac aag uuu aac cac gaa gcc
gaa gac cug uuc uau caa agu uca cuu gcu ucu ugg
aau uau aac acc aau auu acu gaa gag aau guc caa
aac aug aau aac gcu ggg gac aaa ugg ucu gcc uuu
uua aag gaa cag ucc aca cuu gcc caa aug uau cca
cua caa gaa auu cag aau cuc aca guc aag cuu cag
cug cag gcu cuu cag caa aau ggg ucu uca gug cuc
uca gaa gac aag agc aaa cgg uug aac aca auu cua
aau aca aug agc acc auc uac agu acu gga aaa guu
ugu aac cca gau aau cca caa gaa ugc uua uua cuu
gaa cca ggu uug aau gaa aua aug gca aac agu uua
gac uac aau gag agg cuc ugg gcu ugg gaa agc ugg
aga ucu gag guc ggc aag cag cug agg cca uua uau
gaa gag uau gug guc uug aaa aau gag aug gca aga
gca aau cau uau gag gac uau ggg gau uau ugg aga
gga gac uau gaa gua aau ggg gua gau ggc uau gac
uac agc cgc ggc cag uug auu gaa gau gug gaa cau
acc uuu gaa gag auu aaa cca uua uau gaa cau cuu
cau gcc uau gug agg gca aag uug aug aau gcc uau
ccu ucc uau auc agu cca auu gga ugc cuc ccu gcu
cau uug cuu ggu gau aug ugg ggu aga uuu ugg aca
aau cug uac ucu uug aca guu ccc uuu gga cag aaa
cca aac aua gau guu acu gau gca aug gug gac cag
gcc ugg gau gca cag aga aua uuc aag gag gcc gag
aag uuc uuu gua ucu guu ggu cuu ccu aau aug acu
caa gga uuc ugg gaa aau ucc aug cua acg gac cca
gga aau guu cag aaa gca guc ugc cau ccc aca gcu
ugg gac cug ggg aaa ggc gac uuc agg auc cuu aug
ugc aca aag gug aca aug gac gac uuc cug aca gcu
cau cau gag aug ggg cau auu cag uau gau aug gca
uau gcu gca caa ccu uuu cug cua aga aau gga gcu
aau gaa gga uuc cau gaa gcu guu ggg gaa auc aug
uca cuu ucu gca gcc aca ccu aag cau uua aaa ucc
auu ggu cuu cug uca ccc gau uuu caa gaa gac aau
gaa aca gaa aua aac uuc cug cuc aaa caa gca cuc
acg auu guu ggg acu cug cca uuu acu uac aug uua
gag aag ugg agg ugg aug guc uuu aaa ggg gaa auu
ccc aaa gac cag ugg aug aaa aag ugg ugg gag aug
aag cga gag aua guu ggg gug gug gaa ccu gug ccc
cau gau gaa aca uac ugu gac ccc gca ucu cug uuc
cau guu ucu aau gau uac uca uuc auu cga uau uac
aca agg acc cuu uac caa uuc cag uuu caa gaa gca
cuu ugu caa gca gcu aaa cau gaa ggc ccu cug cac
aaa ugu gac auc uca aac ucu aca gaa gcu gga cag
aaa cug uuc aau aug cug agg cuu gga aaa uca gaa
ccc ugg acc cua gca uug gaa aau guu gua gga gca
aag aac aug aau gua agg cca cug cuc aac uac uuu
gag ccc uua uuu acc ugg cug aaa gac cag aac aag
aau ucu uuu gug gga ugg agu acc gac ugg agu cca
uau gca gac caa agc auc aaa gug agg aua agc cua
aaa uca gcu cuu gga gau aga gca uau gaa ugg aac
gac aau gaa aug uac cug uuc cga uca ucu guu gca
uau gcu aug agg cag uac uuu uua aaa gua aaa aau
cag aug auu cuu uuu ggg gag gag gau gug cga gug
gcu aau uug aaa cca aga auc ucc uuu aau uuc uuu
guc acu gca ccu aaa aau gug ucu gau auc auu ccu
aga acu gaa guu gaa aag gcc auc agg aug ucc cgg
agc cgu auc aau gau gcu uuc cgu cug aau gac aac
agc cua gag uuu cug ggg aua cag cca aca cuu gga
ccu ccu aac cag ccc ccu guu ucc uaa

With reference to the above sequence, the first three nucleotides (aug) and the last three nucleotides (uaa) are start and stop codons, respectively. The last three nucleotides can be replaced to uag and uga. The remaining sequence encodes the 1-740 amino acid sequence of hACE2 protein.

### Example 6

mRNA sequence of the hsACE2 variant (1) (SEQ ID NO: 2)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg auc cuu aug ugc aca aag
gug aca aug gac gac uuc cug aca gcu cau cau
gag aug ggg cau auu cag uau gau aug gca uau
gcu gca caa ccu uuu cug cua aga aau gga gcu
aau gaa gga uuc cau gaa gcu guu ggg gaa auc
aug uca cuu ucu gca gcc aca ccu aag cau uua
aaa ucc auu ggu cuu cug uca ccc gau uuu caa
gaa gac aau gaa aca gaa aua aac uuc cug cuc
aaa caa gca cuc acg auu guu ggg acu cug cca
uuu acu uac aug uua gag aag ugg agg ugg aug
guc uuu aaa ggg gaa auu ccc aaa gac cag ugg
aug aaa aag ugg ugg gag aug aag cga gag aua
guu ggg gug gug gaa ccu gug ccc cau gau gaa
aca uac ugu gac ccc gca ucu cug uuc cau guu
ucu aau gau uac uca uuc auu cga uau uac aca
agg acc cuu uac caa uuc cag uuu caa gaa gca
cuu ugu caa gca gcu aaa cau gaa ggc ccu cug
cac aaa ugu gac auc uca aac ucu aca gaa gcu
gga cag aaa cug uuc aau aug cug agg cuu gga
aaa uca gaa ccc ugg acc cua gca uug gaa aau
guu gua gga gca aag aac aug aau gua agg cca
cug cuc aac uac uuu gag ccc uua uuu acc ugg
cug aaa gac cag aac aag aau ucu uuu gug gga
ugg agu acc gac ugg agu cca uau gca gac caa
agc auc aaa gug agg aua agc cua aaa uca gcu
cuu gga gau aga gca uau gaa ugg aac gac aau
gaa aug uac cug uuc cga uca ucu guu gca uau
gcu aug agg cag uac uuu uua aaa gua aaa aau
cag aug auu cuu uuu ggg gag gag gau gug cga
gug gcu aau uug aaa cca aga auc ucc uuu aau
uuc uuu guc acu gca ccu aaa aau gug ucu gau
auc auu ccu aga acu gaa guu gaa aag gcc auc
agg aug ucc cgg agc cgu auc aau gau gcu uuc
cgu cug aau gac aac agc cua gag uuu cug ggg
aua cag cca aca cuu gga ccu ccu aac cag ccc
ccu guu ucc gag aac uug uac uuc caa ucc ggu
aag ccu auc ccu aac ccu cuc cuc ggu cuc gau
ucu acg uaa

With reference to the above sequence, the first three nucleotides (aug) and the last three nucleotides (uaa) are start and stop codons, respectively. The last three nucleotides can be replaced to uag and uga. The sequence corresponding to the TEV site is underlined and is gag aac uug uac uuc caa ucc. The sequence following the TEV site and before the stop codon corresponds to the V5 tag, and is ggu aag ccu auc ccu aac ccu cuc cuc ggu cuc gau ucu acg. The remaining sequence encodes the 1-740 amino acid sequence of hACE2 protein.

### Example 7

mRNA Sequence of the hsACE2 Variant (2) (SEQ ID NO: 3)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes the 1-357 amino acid sequence of hACE2 protein. This truncated variant produced from the above sequence may have the increased accessibility to the spike protein due to a small size, potentially improving binding affinity.

### Example 8

mRNA Sequence of the hsACE2 Variant (3) (SEQ ID NO: 4)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc cuu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg auc cuu aug ugc aca aag
gug aca aug gac gac uuc cug aca gcu cau cau
gag aug ggg cau auu cag uau gau aug gca uau
gcu gca caa ccu uuu cug cua aga aau gga gcu
aau gaa gga uuc cau gaa gcu guu ggg gaa auc
aug uca cuu ucu gca gcc aca ccu aag cau uua
aaa ucc auu ggu cuu cug uca ccc gau uuu caa
gaa gac aau gaa aca gaa aua aac uuc cug cuc
aaa caa gca cuc acg auu guu ggg acu cug cca
uuu acu uac aug uua gag aag ugg agg ugg aug
guc uuu aaa ggg gaa auu ccc aaa gac cag ugg
aug aaa aag ugg ugg gag aug aag cga gag aua
guu ggg gug gug gaa ccu gug ccc cau gau gaa
aca uac ugu gac ccc gca ucu cug uuc cau guu
ucu aau gau uac uca uuc auu cga uau uac aca
agg acc cuu uac caa uuc cag uuu caa gaa gca
cuu ugu caa gca gcu aaa cau gaa ggc ccu cug
cac aaa ugu gac auc uca aac ucu aca gaa gcu
gga cag aaa cug uuc aau aug cug agg cuu gga
aaa uca gaa ccc ugg acc cua gca uug gaa aau
guu gua gga gca aag aac aug aau gua agg cca
cug cuc aac uac uuu gag ccc uua uuu acc ugg
cug aaa gac cag aac aag aau ucu uuu gug gga
ugg agu acc gac ugg agu cca uau gca gac caa
agc auc aaa gug agg aua agc cua aaa uca gcu
cuu gga gau aga gca uau gaa ugg aac gac aau
gaa aug uac cug uuc cga uca ucu guu gca uau
gcu aug agg cag uac uuu uua aaa gua aaa aau
cag aug auu cuu uuu ggg gag gag gau gug cga
gug gcu aau uug aaa cca aga auc ucc uuu aau
uuc uuu guc acu gca ccu aaa aau gug ucu gau
auc auu ccu aga acu gaa guu gaa aag gcc auc
agg aug ucc cgg agc cgu auc aau gau gcu uuc
cgu cug aau gac aac agc cua gag uuu cug ggg
aua cag cca aca cuu gga ccu ccu aac cag ccc
ccu guu ucc uaa

With reference to the above sequence, the first three nucleotides (aug) and the last three nucleotides (uaa) are start and stop codons, respectively. The last three nucleotides can be replaced to uag and uga. The remaining sequence encodes the 1-740 amino acid sequence of hACE2 protein with two mutations. Two mutations are underlined and in bold, and are r105a>c and r109g>a, resulting in pGlu35Asp and pGlu37Lys, respectively. These mutations are incorporated to increase the strength of hydrogen bonds formed with Gln493 and Tyr505 of virus spike protein, which may increase the binding affinity.

### Example 9

mRNA Sequence of the hsACE2 Variant (4) (SEQ ID NO: 5)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes the 1-357 amino acid sequence of hACE2 protein with two mutations. This truncated variant produced from the above sequence may have the increased accessibility to the spike protein due to a small size, potentially resulting in improved binding affinity. Two mutations are underlined and in bold, and are r105a>c and r109g>a, resulting in pGlu35Asp and pGlu37Lys, respectively. These mutations are incorporated to increase the strength of hydrogen bonds formed with Gln493 and Tyr505 of virus spike protein, which may increase the binding affinity.

Example 10

mRNA Sequence of the hsACE2 Variant (5) (SEQ ID NO: 6)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg ggc ggc ggc agc ggc ggc
agc ggc agc ggc ggc agc ggc ggc ggc agc aug
uca agc ucu ucc ugg cuc cuu cuc agc cuu guu
gcu gua acu gcu gcu cag ucc acc auu gag gaa
cag gcc aag aca uuu uug gac aag uuu aac cac
gac gcc aaa gac cug uuc uau caa agu uca cuu
gcu ucu ugg aau uau aac acc aau auu acu gaa
gag aau guc caa aac aug aau aac gcu ggg gac
aaa ugg ucu gcc uuu uua aag gaa cag ucc aca
cuu gcc caa aug uau cca cua caa gaa auu cag
aau cuc aca guc aag cuu cag cug cag gcu cuu
cag caa aau ggg ucu uca gug cuc uca gaa gac
aag agc aaa cgg uug aac aca auu cua aau aca
aug agc acc auc uac agu acu gga aaa guu ugu
aac cca gau aau cca caa gaa ugc uua uua cuu
gaa cca ggu uug aau gaa aua aug gca aac agu
uua gac uac aau gag agg cuc ugg gcu ugg gaa
agc ugg aga ucu gag guc ggc aag cag cug agg
cca uua uau gaa gag uau gug guc uug aaa aau
gag aug gca aga gca aau cau uau gag gac uau
ggg gau uau ugg aga gga gac uau gaa gua aau
ggg gua gau ggc uau gac uac agc cgc ggc cag
uug auu gaa gau gug gaa cau acc uuu gaa gag
auu aaa cca uua uau gaa cau cuu cau gcc uau
gug agg gca aag uug aug aau gcc uau ccu ucc
uau auc agu cca auu gga ugc cuc ccu gcu cau
uug cuu ggu gau aug ugg ggu aga uuu ugg aca
aau cug uac ucu uug aca guu ccc uuu gga cag
aaa cca aac aua gau guu acu gau gca aug gug
gac cag gcc ugg gau gca cag aga aua uuc aag
gag gcc gag aag uuc uuu gua ucu guu ggu cuu
ccu aau aug acu caa gga uuc ugg gaa aau ucc
aug cua acg gac cca gga aau guu cag aaa gca
guc ugc cau ccc aca gcu ugg gac cug ggg aaa
ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes a dimer consisting of two sets of the 1-357 amino acid sequence of hACE2 protein connected by a 16-mer linker sequence. The sequence corresponding to the 16-mer linker is underlined, and is ggc ggc ggc agc ggc ggc agc ggc agc ggc ggc agc ggc ggc ggc agc. The sequence corresponding to the dimer contains four mutations. Four mutations are underlined and in bold, and are r105a>c, r109g>a, r1224a>c, and r1228g>a, resulting in pGlu35Asp, pGlu37Lys, pGlu408Asp, and pGlu410Lys, respectively. The mutations are incorporated to increase the strength of hydrogen bonds formed with Gln493 and Tyr505 of virus spike protein, which may increase the binding affinity. This dimer consisting of two truncated variant produced from the above sequence is bivalent, and therefore has the increased avidity. This dimer may have the increased accessibility to the spike protein due to a small size, potentially improving binding affinity.

### Example 11

mRNA Sequence of the hsACE2 Variant (6) (SEQ ID NO: 7)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg ggc ggc ggc agc ggc ggc
agc ggc agc ggc ggc agc ggc ggc ggc agc aug
uca agc ucu ucc ugg cuc cuu cuc agc cuu guu
gcu gua acu gcu gcu cag ucc acc auu gag gaa
cag gcc aag aca uuu uug gac aag uuu aac cac
gaa gcc gaa gac cug uuc uau caa agu uca cuu
gcu ucu ugg aau uau aac acc aau auu acu gaa
gag aau guc caa aac aug aau aac gcu ggg gac
aaa ugg ucu gcc uuu uua aag gaa cag ucc aca
cuu gcc caa aug uau cca cua caa gaa auu cag
aau cuc aca guc aag cuu cag cug cag gcu cuu
cag caa aau ggg ucu uca gug cuc uca gaa gac
aag agc aaa cgg uug aac aca auu cua aau aca
aug agc acc auc uac agu acu gga aaa guu ugu
aac cca gau aau cca caa gaa ugc uua uua cuu
gaa cca ggu uug aau gaa aua aug gca aac agu
uua gac uac aau gag agg cuc ugg gcu ugg gaa
agc ugg aga ucu gag guc ggc aag cag cug agg
cca uua uau gaa gag uau gug guc uug aaa aau
gag aug gca aga gca aau cau uau gag gac uau
ggg gau uau ugg aga gga gac uau gaa gua aau
ggg gua gau ggc uau gac uac agc cgc ggc cag
uug auu gaa gau gug gaa cau acc uuu gaa gag
auu aaa cca uua uau gaa cau cuu cau gcc uau
gug agg gca aag uug aug aau gcc uau ccu ucc
uau auc agu cca auu gga ugc cuc ccu gcu cau
uug cuu ggu gau aug ugg ggu aga uuu ugg aca
aau cug uac ucu uug aca guu ccc uuu gga cag
aaa cca aac aua gau guu acu gau gca aug gug
gac cag gcc ugg gau gca cag aga aua uuc aag
gag gcc gag aag uuc uuu gua ucu guu ggu cuu
ccu aau aug acu caa gga uuc ugg gaa aau ucc
aug cua acg gac cca gga aau guu cag aaa gca
guc ugc cau ccc aca gcu ugg gac cug ggg aaa
ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes a dimer consisting of two sets of the 1-357 amino acid sequence of hACE2 protein connected by a 16-mer linker sequence. The sequence corresponding to the 16-mer linker is underlined, and is ggc ggc ggc agc ggc ggc agc ggc agc ggc ggc agc ggc ggc ggc agc. This dimer consisting of two truncated variant produced from the above sequence is bivalent, and therefore has the increased avidity. This dimer may have the increased accessibility to the spike protein due to a small size, potentially improving binding affinity.

### Example 12

mRNA Sequence of the hsACE2 Variant (7) (SEQ ID NO: 8)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg ggc ggc agc ggc ggc agc
ggc ggc aug uca agc ucu ucc ugg cuc cuu cuc
agc cuu guu gcu gua acu gcu gcu cag ucc acc
auu gag gaa cag gcc aag aca uuu uug gac aag
uuu aac cac gac gcc aaa gac cug uuc uau caa
agu uca cuu gcu ucu ugg aau uau aac acc aau
auu acu gaa gag aau guc caa aac aug aau aac
gcu ggg gac aaa ugg ucu gcc uuu uua aag gaa
cag ucc aca cuu gcc caa aug uau cca cua caa
gaa auu cag aau cuc aca guc aag cuu cag cug
cag gcu cuu cag caa aau ggg ucu uca gug cuc
uca gaa gac aag agc aaa cgg uug aac aca auu
cua aau aca aug agc acc auc uac agu acu gga
aaa guu ugu aac cca gau aau cca caa gaa ugc
uua uua cuu gaa cca ggu uug aau gaa aua aug
gca aac agu uua gac uac aau gag agg cuc ugg
gcu ugg gaa agc ugg aga ucu gag guc ggc aag
cag cug agg cca uua uau gaa gag uau gug guc
uug aaa aau gag aug gca aga gca aau cau uau
gag gac uau ggg gau uau ugg aga gga gac uau
gaa gua aau ggg gua gau ggc uau gac uac agc
cgc ggc cag uug auu gaa gau gug gaa cau acc
uuu gaa gag auu aaa cca uua uau gaa cau cuu
cau gcc uau gug agg gca aag uug aug aau gcc
uau ccu ucc uau auc agu cca auu gga ugc cuc
ccu gcu cau uug cuu ggu gau aug ugg ggu aga
uuu ugg aca aau cug uac ucu uug aca guu ccc
uuu gga cag aaa cca aac aua gau guu acu gau
gca aug gug gac cag gcc ugg gau gca cag aga
aua uuc aag gag gcc gag aag uuc uuu gua ucu
guu ggu cuu ccu aau aug acu caa gga uuc ugg
gaa aau ucc aug cua acg gac cca gga aau guu
cag aaa gca guc ugc cau ccc aca gcu ugg gac
cug ggg aaa ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes a dimer consisting of two sets of the 1-357 amino acid sequence of hACE2 protein connected by a 8-mer linker sequence. The sequence corresponding to the 8-mer linker is underlined, and is ggc ggc agc ggc ggc agc ggc ggc. The sequence corresponding to the dimer contains four mutations. Four mutations are underlined and in bold, and are r105a>c, r109g>a, r1200a>c, and r1204g>a, resulting in pGlu35Asp, pGlu37Lys, pGlu400Asp, and pGlu402Lys, respectively. The mutations are incorporated to increase the strength of hydrogen bonds formed with Gln493 and Tyr505 of virus spike protein, which may increase the binding affinity. This dimer consisting of two truncated variant produced from the above sequence is bivalent, and therefore has the increased avidity. This dimer may have the increased accessibility to the spike protein due to a small size, potentially improving binding affinity.

### Example 13

mRNA sequence of the hsACE2 variant (8) (SEQ ID NO: 9)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg ggc ggc agc ggc ggc agc
ggc ggc aug uca agc ucu ucc ugg cuc cuu cuc
agc cuu guu gcu gua acu gcu gcu cag ucc acc
auu gag gaa cag gcc aag aca uuu uug gac aag
uuu aac cac gaa gcc gaa gac cug uuc uau caa
agu uca cuu gcu ucu ugg aau uau aac acc aau
auu acu gaa gag aau guc caa aac aug aau aac
gcu ggg gac aaa ugg ucu gcc uuu uua aag gaa
cag ucc aca cuu gcc caa aug uau cca cua caa
gaa auu cag aau cuc aca guc aag cuu cag cug
cag gcu cuu cag caa aau ggg ucu uca gug cuc
uca gaa gac aag agc aaa cgg uug aac aca auu
cua aau aca aug agc acc auc uac agu acu gga
aaa guu ugu aac cca gau aau cca caa gaa ugc
uua uua cuu gaa cca ggu uug aau gaa aua aug
gca aac agu uua gac uac aau gag agg cuc ugg
gcu ugg gaa agc ugg aga ucu gag guc ggc aag
cag cug agg cca uua uau gaa gag uau gug guc
uug aaa aau gag aug gca aga gca aau cau uau
gag gac uau ggg gau uau ugg aga gga gac uau
gaa gua aau ggg gua gau ggc uau gac uac agc
cgc ggc cag uug auu gaa gau gug gaa cau acc
uuu gaa gag auu aaa cca uua uau gaa cau cuu
cau gcc uau gug agg gca aag uug aug aau gcc
uau ccu ucc uau auc agu cca auu gga ugc cuc
ccu gcu cau uug cuu ggu gau aug ugg ggu aga
uuu ugg aca aau cug uac ucu uug aca guu ccc
uuu gga cag aaa cca aac aua gau guu acu gau
gca aug gug gac cag gcc ugg gau gca cag aga
aua uuc aag gag gcc gag aag uuc uuu gua ucu
guu ggu cuu ccu aau aug acu caa gga uuc ugg
gaa aau ucc aug cua acg gac cca gga aau guu
cag aaa gca guc ugc cau ccc aca gcu ugg gac
cug ggg aaa ggc gac uuc agg uaa

With reference to the above sequence, the first three nucleotides (aug) and the last three nucleotides (uaa) are start and stop codons, respectively. The last three nucleotides can be replaced to uag and uga. The remaining sequence encodes a dimer consisting of two sets of the 1-357 amino acid sequence of hACE2 protein connected by a 8-mer linker sequence. The sequence corresponding to the 8-mer linker is underlined, and is ggc ggc agc ggc ggc agc ggc ggc. This dimer consisting of two truncated variant produced from the above sequence is bivalent, and therefore has the increased avidity. This dimer may have the increased accessibility to the spike protein due to a small size, potentially improving binding affinity.

### Example 14

mRNA Sequence of the hsACE2 Variant (9) (SEQ ID NO: 10)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg gaa gcg gcg gcg aaa ggc
uau auu ccg gaa gcg ccg cgc gau ggc cag gcg
uau gug cgc aaa gau ggc gaa ugg gug cug cug
agc acc uuu cug uaa

With reference to the above sequence, the first three nucleotides (aug) and the last three nucleotides (uaa) are start and stop codons, respectively. The last three nucleotides can be replaced to uag and uga. The remaining sequence encodes the 1-357 amino acid sequence of hACE2 protein, followed by a 5-mer linker-foldon fusion protein. This truncated variant produced from the above sequence may have the increased accessibility to the spike protein due to a small size, potentially resulting in improved binding affinity. The sequence corresponding to the 5-mer linker-foldon fusion protein is underlined, and is gaa geg geg geg aaa ggc uau auu ccg gaa geg ccg cgc gau ggc cag geg uau gug cgc aaa gau ggc gaa ugg gug cug cug age acc uuu cug. This variant containing the sequence of the foldon domain, which is derived from the fibritin protein of bacteriophage T4, forms a trimer, which becomes trivalent and therefore has the increased avidity to the viral spike protein.

### Example 15

mRNA Sequence of the hsACE2 Variant (10) (SEQ ID NO: 11)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aac agu uua gac gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg gaa gcg gcg gcg aaa ggc
uau auu ccg gaa gcg ccg cgc gau ggc cag gcg
uau gug cgc aaa gau ggc gaa ugg gug cug cug
agc acc uuu cug uaa

With reference to the above sequence, the first three nucleotides (aug) and the last three nucleotides (uaa) are start and stop codons, respectively. The last three nucleotides can be replaced to uag and uga. The remaining sequence encodes the 1-357 amino acid sequence of hACE2 protein with two mutations, followed by a 5-mer linker-foldon fusion protein. Two mutations are underlined, and are r105a>c and r109g>a, resulting in pGlu35Asp and pGlu37Lys, respectively. These mutations are incorporated to increase the strength of hydrogen bonds formed with Gln493 and Tyr505 of virus spike protein, which may increase the binding affinity. This truncated variant produced from the above sequence may have the increased accessibility to the spike protein due to a small size, potentially resulting in improved binding affinity. The sequence corresponding to the 5-mer linker-foldon fusion protein is underlined, and is gaa geg geg geg aaa ggc uau auu ccg gaa geg ccg cgc gau ggc cag geg uau gug cgc aaa gau ggc gaa ugg gug cug cug agc acc uuu cug. This variant containing the sequence of the foldon domain, which is derived from the fibritin protein of bacteriophage T4, forms a trimer, which becomes trivalent and therefore has the increased avidity to the viral spike protein.

### Example 16

mRNA Sequence of the hsACE2 Variant (11) (SEQ ID NO: 12)

aug uca agc ucu ucc ugg cuc cuu cuc agc cuu
guu gcu gua acu gcu gcu cag ucc acc auu gag
gaa cag gcc aag aca uuu uug gac aag uuu aac
cac gaa gcc gaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg auc cuu aug ugc aca aag
gug aca aug gac gac uuc cug aca gcu cau cau
gag aug ggg cau auu cag uau gau aug gca uau
gcu gca caa ccu uuu cug cua aga aau gga gcu
aau gaa gga uuc cau gaa gcu guu ggg gaa auc
aug uca cuu ucu gca gcc aca ccu aag cau uua
aaa ucc auu ggu cuu cug uca ccc gau uuu caa
gaa gac aau gaa aca gaa aua aac uuc cug cuc
aaa caa gca cuc acg auu guu ggg acu cug cca
uuu acu uac aug uua gag aag ugg agg ugg aug
guc uuu aaa ggg gaa auu ccc aaa gac cag ugg
aug aaa aag ugg ugg gag aug aag cga gag aua
guu ggg gug gug gaa ccu gug ccc cau gau gaa
aca uac ugu gac ccc gca ucu cug uuc cau guu
ucu aau gau uac uca uuc auu cga uau uac aca
agg acc cuu uac caa uuc cag uuu caa gaa gca
cuu ugu caa gca gcu aaa cau gaa ggc ccu cug
cac aaa ugu gac auc uca aac ucu aca gaa gcu
gga cag aaa cug uuc aau aug cug agg cuu gga
aaa uca gaa ccc ugg acc cua gca uug gaa aau
guu gua gga gca aag aac aug aau gua agg cca
cug cuc aac uac uuu gag ccc uua uuu acc ugg
cug aaa gac cag aac aag aau ucu uuu gug gga
ugg agu acc gac ugg agu cca uau gca gac caa
agc auc aaa gug agg aua agc cua aaa uca gcu
cuu gga gau aga gca uau gaa ugg aac gac aau
gaa aug uac cug uuc cga uca ucu guu gca uau
gcu aug agg cag uac uuu uua aaa gua aaa aau
cag aug auu cuu uuu ggg gag gag gau gug cga
gug gcu aau uug aaa cca aga auc ucc uuu aau
uuc uuu guc acu gca ccu aaa aau gug ucu gau
auc auu ccu aga acu gaa guu gaa aag gcc auc
agg aug ucc cgg agc cgu auc aau gau gcu uuc
cgu cug aau gac aac agc cua gag uuu cug ggg
aua cag cca aca cuu gga ccu ccu aac cag ccc
ccu guu ucc gaa gcg gcg gcg aaa ggc uau auu
ccg gaa gcg ccg cgc gau ggc cag gcg uau gug
cgc aaa gau ggc gaa ugg gug cug cug agc acc
uuu cug uaa

With reference to the above sequence, the first three nucleotides (aug) and the last three nucleotides (uaa) are start and stop codons, respectively. The last three nucleotides can be replaced to uag and uga. The remaining sequence encodes the 1-740 amino acid sequence of hACE2 protein, followed by a 5-mer linker-foldon fusion protein. The sequence corresponding to the 5-mer linker-foldon fusion protein is underlined, and is gaa geg geg geg aaa gge uau auu ccg gaa geg ccg cgc gau ggc cag gcg uau gug cgc aaa gau ggc gaa ugg gug cug cug agc acc uuu cug. This variant containing the sequence of the foldon domain, which is derived from the fibritin protein of bacteriophage T4, forms a trimer, which becomes trivalent and therefore has the increased avidity to the viral spike protein.

### Example 17

mRNA Sequence of the hsACE2 Variant (12) (SEQ ID NO: 13)

aug uca agc ucu ucc ugg cuc cuu cuc agc cuu
guu gcu gua acu gcu gcu cag ucc acc auu gag
gaa cag gcc aag aca uuu uug gac aag uuu aac
cac gac gcc aaa gac cug uuc uau caa agu uca
cuu gcu ucu ugg aau uau aac acc aau auu acu
gaa gag aau guc caa aac aug aau aac gcu ggg
gac aaa ugg ucu gcc uuu uua aag gaa cag ucc
aca cuu gcc caa aug uau cca cua caa gaa auu
cag aau cuc aca guc aag cuu cag cug cag gcu
cuu cag caa aau ggg ucu uca gug cuc uca gaa
gac aag agc aaa cgg uug aac aca auu cua aau
aca aug agc acc auc uac agu acu gga aaa guu
ugu aac cca gau aau cca caa gaa ugc uua uua
cuu gaa cca ggu uug aau gaa aua aug gca aac
agu uua gac uac aau gag agg cuc ugg gcu ugg
gaa agc ugg aga ucu gag guc ggc aag cag cug
agg cca uua uau gaa gag uau gug guc uug aaa
aau gag aug gca aga gca aau cau uau gag gac
uau ggg gau uau ugg aga gga gac uau gaa gua
aau ggg gua gau ggc uau gac uac agc cgc ggc
cag uug auu gaa gau gug gaa cau acc uuu gaa
gag auu aaa cca uua uau gaa cau cuu cau gcc
uau gug agg gca aag uug aug aau gcc uau ccu
ucc uau auc agu cca auu gga ugc cuc ccu gcu
cau uug cuu ggu gau aug ugg ggu aga uuu ugg
aca aau cug uac ucu uug aca guu ccc uuu gga
cag aaa cca aac aua gau guu acu gau gca aug
gug gac cag gcc ugg gau gca cag aga aua uuc
aag gag gcc gag aag uuc uuu gua ucu guu ggu
cuu ccu aau aug acu caa gga uuc ugg gaa aau
ucc aug cua acg gac cca gga aau guu cag aaa
gca guc ugc cau ccc aca gcu ugg gac cug ggg
aaa ggc gac uuc agg auc cuu aug ugc aca aag
gug aca aug gac gac uuc cug aca gcu cau cau
gag aug ggg cau auu cag uau gau aug gca uau
gcu gca caa ccu uuu cug cua aga aau gga gcu
aau gaa gga uuc cau gaa gcu guu ggg gaa auc
aug uca cuu ucu gca gcc aca ccu aag cau uua
aaa ucc auu ggu cuu cug uca ccc gau uuu caa
gaa gac aau gaa aca gaa aua aac uuc cug cuc
aaa caa gca cuc acg auu guu ggg acu cug cca
uuu acu uac aug uua gag aag ugg agg ugg aug
guc uuu aaa ggg gaa auu ccc aaa gac cag ugg
aug aaa aag ugg ugg gag aug aag cga gag aua
guu ggg gug gug gaa ccu gug ccc cau gau gaa
aca uac ugu gac ccc gca ucu cug uuc cau guu
ucu aau gau uac uca uuc auu cga uau uac aca
agg acc cuu uac caa uuc cag uuu caa gaa gca
cuu ugu caa gca gcu aaa cau gaa ggc ccu cug
cac aaa ugu gac auc uca aac ucu aca gaa gcu
gga cag aaa cug uuc aau aug cug agg cuu gga
aaa uca gaa ccc ugg acc cua gca uug gaa aau
guu gua gga gca aag aac aug aau gua agg cca
cug cuc aac uac uuu gag ccc uua uuu acc ugg
cug aaa gac cag aac aag aau ucu uuu gug gga
ugg agu acc gac ugg agu cca uau gca gac caa
agc auc aaa gug agg aua agc cua aaa uca gcu
cuu gga gau aga gca uau gaa ugg aac gac aau
gaa aug uac cug uuc cga uca ucu guu gca uau
gcu aug agg cag uac uuu uua aaa gua aaa aau
cag aug auu cuu uuu ggg gag gag gau gug cga
gug gcu aau uug aaa cca aga auc ucc uuu aau
uuc uuu guc acu gca ccu aaa aau gug ucu gau
auc auu ccu aga acu gaa guu gaa aag gcc auc
agg aug ucc cgg agc cgu auc aau gau gcu uuc
cgu cug aau gac aac agc cua gag uuu cug ggg
aua cag cca aca cuu gga ccu ccu aac cag ccc
ccu guu ucc gaa gcg gcg gcg aaa ggc uau auu
ccg gaa gcg ccg cgc gau ggc cag gcg uau gug
cgc aaa gau ggc gaa ugg gug cug cug agc acc
uuu cug uaa

With reference to the above sequence, the first three nucleotides (aug) and the last three nucleotides (uaa) are start and stop codons, respectively. The last three nucleotides can be replaced to uag and uga. The remaining sequence encodes the 1-740 amino acid sequence of hACE2 protein with two mutations, followed by a 5-mer linker-foldon fusion protein. Two mutations are underlined, and are r105a>c and r109g>a, resulting in pGlu35Asp and pGlu37Lys, respectively. These mutations are incorporated to increase the strength of hydrogen bonds formed with Gln493 and Tyr505 of virus spike protein, which may increase the binding affinity. The sequence corresponding to the 5-mer linker-foldon fusion protein is underlined, and is gaa gcg gcg gcg aaa ggc uau auu ccg gaa geg ccg cgc gau ggc cag gog uau gug cgc aaa gau ggc gaa ugg gug cug cug agc acc uuu cug. This variant containing the sequence of the foldon domain, which is derived from the fibritin protein of bacteriophage T4, forms a trimer, which becomes trivalent and therefore has the increased avidity to the viral spike protein.

With reference to the above Examples 5-17, it may be understood that the present disclosure also encompasses the amino acid sequences corresponding to each of the mRNA sequences disclosed.

It may be understood that the methods laid out in this disclosure are not limited to soluble forms of the hACE2 protein, but encompass other receptors (or portions/variations thereof) which are recognized by viral proteins, bacterial proteins, and the like, capable of causing disease or other health complications via their interaction with such receptors.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method of treating a patient suffering from a condition or disease, comprising:
administering to the patient an effective amount of a therapeutic agent comprising one or more RNA molecules encapsulated by a lipid nanoparticle;
wherein said treating of the patient reduces at least one or more signs or symptoms associated with the condition or disease.

2. The method of claim 1, wherein the RNA is mRNA, and encodes for a soluble form of human angiotensin-converting enzyme 2 (hACE2) and/or one or more variations thereof.

3. The method of claim 1, wherein the condition or disease is a viral infection.

4. The method of claim 3, wherein the viral infection is caused by severe acute respiratory syndrome coronavirus 2 (SARS-COV-2).

5. The method of claim 1, wherein the lipid nanoparticle is comprised of an ionizable lipid, a PEG lipid, a sterol or substitution thereof, and a structural lipid.

6. The method of claim 5, wherein the lipid nanoparticle does not include cholesterol.

7. The method of claim 5, wherein the sterol of the lipid nanoparticle is β-sitosterol.

8. The method of claims 1, wherein the therapeutic agent is administered to the patient intravenously.

9. The method of claims 1, wherein the therapeutic agent is administered to the patient by inhalation.

10. The method of claim 2, wherein an expression of the soluble form of hACE2 and/or one or more variations thereof is dependent on a dosage of the therapeutic agent.

11. The method of claim 10, wherein the expression of the soluble form of hACE2 and/or one or more variations thereof is time-dependent with a highest level of expression around 6 hours after the administration of the therapeutic agent.

12. A therapeutic agent for treating a patient suffering from a viral infection, comprising:
a lipid nanoparticle comprised of each of an ionizable lipid, a PEG lipid, β-sitosterol, and a structural lipid; and
one or more mRNA molecules encoding at least a portion of a soluble protein encapsulated within the lipid nanoparticle.

13. The therapeutic agent of claim 12, wherein the lipid nanoparticle does not include cholesterol.

14. The therapeutic agent of claim 12, wherein the mRNA encodes for a soluble form of human angiotensin-converting enzyme 2 (hACE2), and/or one or more variations thereof.

15. The therapeutic agent of claim 12, wherein the viral infection is caused by severe acute respiratory syndrome coronavirus 2 (SARS-COV-2).

16. The therapeutic agent of claim 14, wherein the mRNA encoding for the soluble form of hACE2 and/or one or more variations thereof comprises one or more sequences of SEQ ID NOs: 1-13.

17. The therapeutic agent of claim 14, wherein the soluble form of hACE2 and/or one or more variations thereof bind to a receptor-binding domain of a spike protein of the virus with a high affinity.

18. The therapeutic agent of claim 14, wherein the soluble form of hACE2 and/or one or more variations thereof reduce the viral infection through competitive inhibition.

19. The therapeutic agent of claim 12, wherein the ionizable lipid comprises one or more of DLin-KC2-DMA, DLin-MC3-DMA, C12-200, cKK-E12, L319, YSK12-C4, YSK05, CL4H6, SM-102, Lipid 9, Lipid 5, ALC-0315, DOTAP, DODAP, DODMA, TT3, LP01, and Lipid 10.

20. The therapeutic agent of claim 12, wherein the PEG lipid comprises one or more of DMG-PEG, DSG-PEG, DPG-PEG, DSPE-PEG, DPPE-PEG, DMPE-PEG, 14:0 PEG, and ALC-0159.

21. The therapeutic agent of claim 12, wherein the structural lipid comprises one or more of 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dipalmitoyl-sn-glycero-3-phosphocholine (DPPC), 1,2-dipalmitoyl-sn-glycero-3-phosphoethanolamine (DPPE), 1,2-dimyristoyl-sn-glycero-3-phosphoethanolamine (DMPE), and 1,2-dioleoyl-sn-glycero-3-phospho-(1′-rac-glycerol) (DOPG).

22. A method of treating a patient suffering from an infection caused by severe acute respiratory syndrome coronavirus 2 (SARS-COV-2), comprising:
administering to the patient an effective amount of a therapeutic agent comprising one or more mRNA molecules encoding at least a portion of a soluble form of human angiotensin-converting enzyme 2 (hACE2) encapsulated by a lipid nanoparticle, the lipid nanoparticle including an ionizable lipid, a PEG lipid, β-sitosterol, and a structural lipid;
wherein said treating of the patient reduces at least one or more signs or symptoms associated with the infection.

23. The method of claim 22, wherein the therapeutic agent is administered to the patient intravenously in a single dose or in multiple doses.

24. The method of claim 22, wherein the therapeutic agent is administered to the patient by inhalation in a single dose or in multiple doses.

25. The method of claim 22, wherein the one or more mRNA molecules comprise one or more sequences of SEQ ID NOs: 1-13.

26. The method of claim 22, wherein the soluble form of hACE2 binds to a receptor-binding domain of a spike protein of SARS-COV-2, and wherein the soluble form of hACE2 reduces the one or more signs or symptoms associated with the infection through a competitive inhibition of SARS-COV-2.


SEQUENCE LISTING (13 sequences)

SEQ ID NO: 1 - RNA, 2223 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggauccuuaugugcacaaaggugacaauggacgacuuccugacagcucaucaugagauggggcauauucaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauagagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccuaa

SEQ ID NO: 2 - RNA, 2286 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggauccuuaugugcacaaaggugacaauggacgacuuccugacagcucaucaugagauggggcauauucaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauagagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccgagaacuuguacuuccaauccgguaagccuaucccuaacccucuccucggucucgauucuacguaa

SEQ ID NO: 3 - RNA, 1074 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 4 - RNA, 2223 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggauccuuaugugcacaaaggugacaauggacgacuuccugacagcucaucaugagauggggcauauucaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauagagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccuaa

SEQ ID NO: 5 - RNA, 1074 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 6 - RNA, 2193 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggggcggcggcagcggcggcagcggcagcggcggcagcggcggcggcagcaugucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 7 - RNA, 2193 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggggcggcggcagcggcggcagcggcagcggcggcagcggcggcggcagcaugucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 8 - RNA, 2169 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggggcggcagcggcggcagcggcggcaugucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 9 - RNA, 2169 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggggcggcagcggcggcagcggcggcaugucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagguaa

SEQ ID NO: 10 - RNA, 1170 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagggaagcggcggcgaaaggcuauauuccggaagcgccgcgcgauggccaggcguaugugcgcaaagauggcgaaugggugcugcugagcaccuuucuguaa

SEQ ID NO: 11 - RNA, 1170 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucagggaagcggcggcgaaaggcuauauuccggaagcgccgcgcgauggccaggcguaugugcgcaaagauggcgaaugggugcugcugagcaccuuucuguaa

SEQ ID NO: 12 - RNA, 2319 nt, Artificial Sequence
  Synthetic
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggauccuuaugugcacaaaggugacaauggacgacuuccugacagcucaucaugagauggggcauauucaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauagagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccgaagcggcggcgaaaggcuauauuccggaagcgccgcgcgauggccaggcguaugugcgcaaagauggcgaaugggugcugcugagcaccuuucuguaa

SEQ ID NO: 13 - RNA, 2319 nt, Artificial Sequence
  Synthetic
augucaagcucuuccuggcuccuucucagccuuguugcuguaacugcugcucaguccaccauugaggaacaggccaagacauuuuuggacaaguuuaaccacgacgccaaagaccuguucuaucaaaguucacuugcuucuuggaauuauaacaccaauauuacugaagagaauguccaaaacaugaauaacgcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaaaggcgacuucaggauccuuaugugcacaaaggugacaauggacgacuuccugacagcucaucaugagauggggcauauucaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauagagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccgaagcggcggcgaaaggcuauauuccggaagcgccgcgcgauggccaggcguaugugcgcaaagauggcgaaugggugcugcugagcaccuuucuguaa